(19) United States
(12) Patent Application Publication
(10) Pub. No.: US 2011/0257176 A1
(43) Pub. Date: Oct. 20, 2011

(54) NITROGEN HETEROCYCLE DERIVATIVES AS PROTEASOME MODULATORS

(75) Inventors: Bruno VILLOUTREIX, Paris (FR); Michele REBOUD-RAVAUX, Paris (FR); Nicolas BASSE, Cambridge (GB); Joelle VIDAL, Rennes (FR); Matthieu MONTES, Paris (FR)

(73) Assignees: INSTITUT NATIONAL DE LA SANTE ET DE LA RECHERCHE MEDICALE (INSERM), Paris (FR); CENTRE NATIONAL DE LA RECHERCHE SCIENTIFIQUE, Paris (FR); UNIVERSITE RENNES 1, Rennes (FR); UNIVERSITE PARIS DIDEROT, Paris (FR); UNIVERSITE PIERRE ET MARIE CURIE, Paris (FR)

(21) Appl. No.: 13/079,454

(22) Filed: Apr. 4, 2011

Related U.S. Application Data
(63) Continuation-in-part of application No. 13/001,733.

(30) Foreign Application Priority Data
Jul. 4, 2008 (EP) 08305377.7

(51) Int. Cl. A61K 31/5377; A61K 31/4245; A61K 31/4192; A61K 31/4196; A61P 17/00; A61P 35/00; A61P 37/00; A61P 33/00; A61P 31/12; A61P 31/04; A61P 29/00; A61P 9/00; A61P 17/02; A61P 21/00; A61K 31/41

(52) U.S. Cl. 514/236.2; 514/359; 514/364; 514/383; 514/384

(57) ABSTRACT

A method for treating and/or ameliorating and/or preventing a disease or a disorder, the method comprising administering to an individual in need thereof at least one nitrogen heterocycle derivative of formula (I):

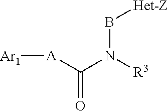

The at least one nitrogen heterocycle derivative may also be used as a proteasome activity modulator in the manufacture of a pharmaceutical composition intended to prevent and/or treat a disease condition mediated by the proteasome activity.

### CROSS-REFERENCE TO RELATED APPLICATIONS

This is a Continuation-in-Part of U.S. application Ser. No. 13/001,733 filed Dec. 28, 2010, which in turn is the U.S. National Phase application of International Application PCT/IB2009/0052918 filed on Jul. 3, 2009, which claims the benefits of European Patent Application No. 08305377.7 filed on Jul. 4, 2008. The disclosures of the prior applications are hereby incorporated by reference herein in their entireties.

### TECHNICAL FIELD

The present invention relates to the identification and the use of nitrogen heterocycle derivatives, such as oxadiazole and triazole derivatives, as modulators of the proteasome activity. More particularly, the invention relates to the use of oxadiazole derivatives for the preparation of pharmaceutical compositions or in cosmetic compositions. The present invention is also directed to a method of prevention and/or treatment of disease conditions mediated by proteasome activity, in particular cancer conditions, comprising the administration of nitrogen heterocycle derivatives, in particular triazole or oxadiazole derivatives in accordance with the invention.

### BACKGROUND

In eukaryotes, the non-lysosomal protein degradation is mainly performed by the strictly controlled complex enzymatic machinery of the ubiquitin-proteasome pathway. Proteasomes are involved in protein quality control and turnover of many critical proteins participating in a vast number of essential biological processes, such as signal transduction, cell proliferation, cell cycle control, cell differentiation and apoptosis (Coux et al., Annu Rev Biochem 1996, 65, 801; Ciechanover et al., Proc Natl Acad Sci USA 1998, 95:2727).

The 26S proteasome is formed by the 20S catalytic core, capped at each end by a regulatory component termed the 19S complex, responsible in part for the selective degradation of a given substrate.

The 20S proteasome core particle is composed of 28 subunits that are arranged into four stacked rings of seven α-type subunits or seven β-type subunits.

The central proteolytic chamber of this endoprotease is composed of the two β-rings. Each β-ring has three different proteolytic activities associated to a N-terminal threonine hydrolase, referred to as chymotrypsin-like (CT-L), post glutamyl peptide hydrolysing or post-acid (PGPH or PA) and trypsin-like (T-L).

Because the proteasome is a universal and broadly active cellular component, it is not surprising that it has been proposed as an interesting target in many disease indications. For instance, the proteasome plays a key role in immune surveillance against virus and cancer, because it is involved in antigen processing and presentation to cytotoxic T cells, and in activation of nuclear factor-kappa B that is the central transcription factor of the immune system. It plays also a role in inflammatory responses, such as inflammatory arthritis, muscle atrophy, in several dystrophies such as limb-girdle muscular dystrophy LGMD-1C) and Duchenne muscular dystrophy (DMD), as well as sleeping sickness.

The proteasome is also proposed as target for cancer therapy, as cancer cells are more susceptible to undergo apoptosis than normal cells after treatment with proteasome inhibitors (Adams et al., Cancer Cell, 2004, 5:417). Proteasome inhibitors sensitize cancer cells and tumours to the proapoptotic effects of conventional chemotherapeutics and radiation therapy (Yu et al., Mol Cancer Ther, 2006, 5:2378.

Several classes of proteasome inhibitors have been described, most of these inhibitors are peptidic derivatives or natural compounds, but the large majority of these molecules is bearing a reactive group and acts as covalent inhibitors. Typically, proteasome inhibitors are sorted according to their ability to interact covalently or non-covalently with the active sites of the proteasome. The different classes of proteasome inhibitors are reviewed by Borissenko & Groll (Chemical Rev., 2007, 107:687) and Papapostolou & Reboud-Ravaux (J Soc Biol, 2004, 198:263).

For example, Velcade® or bortezomib, which is the first proteasome inhibitor approved by the FDA, is a covalently interacting proteasome inhibitor. This compound is a dipeptide boronic acid which is sensitive to oxidation (Pekol et al., Drug Metab Dispos 2005, 33:771.).

The natural compound TMC-95 is a non-covalently interacting proteasome inhibitor (Konno et al., J Org Chem 2000, 65:990). However, this compound presents the drawbacks of having a very complex structure, rendering complicated its synthesis, and a high molecular weight, conferring to it a weak bioavailability.

On the other side, proteasome activators may be useful for improving, treating and/or preventing conditions mediated by accumulation of proteins or polypeptides.

For example, the activation of proteasome should accelerate and improve the intracellular proteolysis favoring the removing of oxidized protein associated with aging, in particular skin aging. Proteasome activators or inhibitors would also be helpful in disease conditions associated with proteins accumulation such as Alzheimer disease or Parkinson disease.

Proteasome activators are, for example, described in Kisselev et al., (J. Biol. Chem. 2002, 277:22260); Wilk et al., (Mol. Biol. Rep., 1997,24:119); Ruiz de Mena et al., (Biochem. J. 1993, 296:93); Arribas et al., (J. Biol. Chem., 1990, 265:13969); U.S. Pat. No. 5,847,076 and JP 2002-029996.

So far, the known proteasome modulators (inhibitors or activators) present a peptide-like structure and/or have a high molecular weight. This features usually leads to a weak bioavailability because they may result in a high degradation rate and/or a low capacity to cross the biological membrane.

Besides, the proteasome modulators that covalently interact with proteasome active sites are typically associated with numerous unwanted side effects. Their reactive group is inherently associated with lack of specifity, excessive reactivity, and instability.

Therefore, there is a need to have novel proteasome activity modulators, and in particular proteasome activity inhibitors, that are selective to the proteasome with respect to the other intracellular proteases.

There is a need for novel proteasome activity modulators, and in particular proteasome activity inhibitors, having a non-peptide like structure.

There is also a need for novel proteasome activity modulators, and in particular proteasome activity inhibitors, having a low molecular weight.

There is also a need for novel proteasome activity modulators, and in particular proteasome activity inhibitors, having a high bioavailability.

There is also a need for novel proteasome modulators, and in particular proteasome activity inhibitors, that non-covalently interact with the active site of the proteasome.

There is also a need for novel proteasome activity modulators, and in particular proteasome activity inhibitors, having reduced or no unwanted side effects.

The present invention has for object to meet those needs.

### SUMMARY

A method for treating and/or ameliorating and/or preventing a disease or a disorder, the method comprising administering to an individual in need thereof at least one nitrogen heterocycle derivative of formula (I):

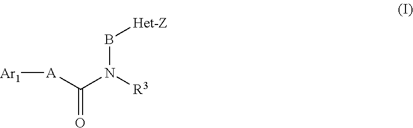

wherein

Het represents a triazole or an oxadiazole radical, optionally substituted with one or more linear or branched, saturated or unsaturated C₁-C₄ alkyl group,

Ar₁ represents a C₆-C₁₀ aryl group, substituted with at least one R group chosen among:
H,
an halogen group, or
a linear, branched or cyclic, saturated or unsaturated, C₁-C₅ alkyl group, or
a linear, branched or cyclic, saturated or unsaturated, C₁-C₅ alkoxy group,
a phenoxy group,

A represents:
a covalent bond, or
*-X—C(R⁴R⁶)-□,
with *- representing a covalent bond with Ar₁, -□ representing a covalent bond with —C(O)—, X representing a linear or branched, saturated or unsaturated, C₁-C₅ alkylene group, or an heteroatom, and R⁴ and R⁶ being, independently of each other chosen among H or a linear, branched or cyclic, saturated or unsaturated, C₁-C₅ alkyl group, or
a linear, branched or cyclic, saturated or unsaturated, C₁-C₅ alkylene group,

B represents a linear, branched or cyclic, saturated or unsaturated, C₁-C₅ alkylene group, optionally substituted with one or more C₁-C₅ hydroxyalkyl group(s), or a C₆-C₁₀ arylene group,

R³ represents H or a linear, branched or cyclic, saturated or unsaturated, C₁-C₅ alkyl group, and

Z represents —(R₅)ₙ—(Ar₂)ₘ, with n and m represent, independently of each other, 0 or 1, provided that at least one of n or in is 1, where
R⁵ represents, a linear, branched or cyclic, saturated or unsaturated, C₁-C₆, alkyl or alkylamido group, optionally comprising one or more heteroatom(s) chosen among O, N or S, and being optionally substituted with one or more halogen group(s), and
Ar₂ represents a C₆-C₁₀ aryl group substituted with at least one R as above-defined.

In embodiments, the at least one nitrogen heterocycle derivative is used as proteasome activity modulator in the manufacture of a pharmaceutical composition intended to prevent and/or treat a disease condition mediated by the proteasome activity.

### DETAILED DESCRIPTION OF EMBODIMENTS

The inventors have surprisingly identified nitrogen heterocycle derivatives, and in particular oxadiazole and triazole derivatives as novel proteasome activity modulators within a compound collection known and commercially available at ChemBridge Corporation on (www.chembridge.com).

The compounds of the invention may also be prepared according to methods well-known by the skilled artisan.

Within the meaning of the invention, “proteasome activity modulator” or “proteasome modulator” are used interchangeably and are intended to mean a compound able to bind with at least one active site of the proteasome, i.e. a chymotrypsin-like active site, a trypsin-like active site or a post-acid (PA) active site or with a site distant from said active sites, and to, directly or allosterically, reduce or even suppress, or increase the enzymatic activity rate of at least one active site as compared with said enzymatic activity rate without said proteasome modulator.

A proteasome modulator of the invention may exert a dual or a multiple effect, that is it may exert an effect on more than one active site.

For example, a proteasome modulator may simultaneously exert a reduction, or a suppression, of the enzymatic activity rate of a first active site and an increase or a decrease of the enzymatic activity rate of a second active site. The enzymatic activity rate of a third active site may be reduced or suppressed, or increased or left unchanged.

A proteasome modulator may be a “proteasome activity inhibitor or proteasome inhibitor”.

Within the meaning of the invention, a “a proteasome activity modulator” or “proteasome activity inhibitor” or “proteasome inhibitor” is intended to mean a proteasome modulator that reduces or suppresses the enzymatic activity rate of at least one active site of the proteasome, as compared with said enzymatic activity rate determined without said proteasome inhibitor.

A proteasome modulator may be a “proteasome activity activator” or “proteasome activator”.

Within the meaning of the invention, a “proteasome activity activator” or a “proteasome activator” is intended to mean a proteasome modulator that increases the enzymatic activity rate of at least one proteasome active site, as compared with said enzymatic activity rate determined without said proteasome activator.

According to one embodiment, a proteasome modulator may exert only an inhibitory effect.

According to one embodiment, a proteasome modulator may exert only an activator effect.

According to one embodiment, a proteasome modulator may exert a dual or a multiple effect.

According to another embodiment, a proteasome modulator may exert an effect on one, two or three active sites.

A proteasome modulator exerting an effect on no more than two, and in particular on no more than one active site may advantageously allow to reduce the risk of occurrence of toxic or unwanted side effects.

According to one embodiment, a proteasome inhibitor of the invention may inhibit no more than two active sites, and in particular no more than one active site.

According to one embodiment, a proteasome inhibitor may advantageously exert an inhibiting effect on the chymotrypsin-like and/or trypsin-like effect, and in particular on the chymotrypsin-like active site.

Within the invention, the terms “inhibitor” or “activator” may be attributed to a compound of the invention with respect to an enzyme activity. However, it does not preclude that said compound may also exert an inhibitor and/or activator effect(s) on the other(s) enzyme activity(ies) of the proteasome.

Within the meaning of the invention, “pharmaceutical composition” is intended to mean any substance or composition intended to be administered to an individual, human or animal, to prevent, reduce, relieve and/or cure a disease condition or a sign associated with said disease condition and/or to make a diagnostic of a disease condition.

Within the meaning of the invention, the term “prevent” or “prevention” with respect to an event is intended to mean the decrease of a risk of occurrence of said event.

According to one embodiment, a disease condition considered in the invention may be chosen among cancers, immunological diseases, auto-immune diseases, allograft rejections, viral diseases, such as mumps, measles, Rous sarcoma or AIDS, parasitic diseases such as malaria or trypanosome, bacterial infections, such as tuberculosis, inflammatory diseases, such as polyarthritis or liver inflammation, cardiac diseases and ischemic strokes, such as myocardial, cerebral or pulmonary ischemic injuries, muscular dystrophies, muscle wasting, traumatisms, burns, disease conditions associated with aging, such as neurodegenerative diseases.

According to another aspect, the invention is directed to a use of at least one nitrogen heterocycle derivative, in particular one oxadiazole or one triazole derivative, in accordance with the invention as active agent in a cosmetic composition for the prevention and/or the treatment of skin aging.

According to another of its aspect, the invention is directed to a kit-of-parts comprising (i) at least one nitrogen heterocycle derivative, in particular an oxadiazole or a triazole derivative, according to the invention and (ii) at least one agent useful for the prevention and/or the treatment of a cancer condition, said agent being different of said nitrogen heterocycle derivative (i).

According to another of its aspect, the invention is directed to a method for preventing and/or treating a disease condition mediated by proteasome activity comprising at least a step of administering to an individual in need thereof at least one effective amount of at least one nitrogen heterocycle derivative, in particular an oxadiazole or a triazole derivative, according to the invention.

According to another of its aspect, the invention is directed a nitrogen heterocycle derivative of the invention for use as a medicament.

According to one advantage, the novel proteasome modulators of the invention have an improved bioavailability.

According to another advantage, the novel proteasome modulators of the invention have an improved cellular toxicity towards tumoral cells.

According to another advantage, the novel proteasome modulators of the invention have a low or even have no cellular toxicity on normal healthy cells.

According to another advantage, the novel proteasome modulators of the invention have reduced or no unwanted side-effects.

According to another advantage, the proteasome modulators of the invention are non peptidic molecules without reactive group susceptible to lead to a lack of specificity, excessive reactivity and instability.

Proteasome Modulators

A nitrogen heterocycle derivative of the invention may be of the following formula (I):

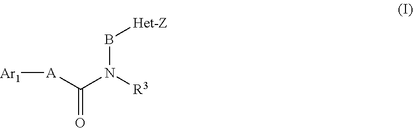

wherein

Het represents a triazole or an oxadiazole radical, optionally substituted with one or more, linear or branched, saturated or unsaturated, C₁-C₄ alkyl group,

Ar₁ represents a C₆-C₁₀ aryl group, substituted with at least one R group chosen among:
H,
an halogen group, or
a linear, branched or cyclic, saturated or unsaturated, C₁-C₅ alkyl group, or
a linear, branched or cyclic, saturated or unsaturated, C₁-C₅ alkoxy group,
a phenoxy group,

A represents:
a covalent bond, or
*—X—C(R⁴R⁶)-□,
with *- representing a covalent bond with Ar₁, -□ representing a covalent bond with —C(O)—, X representing a linear or branched, saturated or unsaturated, C₁-C₅ alkylene group, or an heteroatom, and R⁴ and R⁶ being, independently of each other, chosen among H or a linear, branched or cyclic, saturated or unsaturated, C₁-C₅ alkyl group, or
a linear, branched or cyclic, saturated or unsaturated, C₁-C₅ alkylene group,

B represents a linear, branched or cyclic, saturated or unsaturated, C₁-C₅ alkylene group, optionally substituted with one or more C₁-C₅ hydroxyalkyl group(s), or a C₆-C₁₀ arylene group,

R³ represents H or a linear, branched or cyclic, saturated or unsaturated, C₁-C₅ alkyl group,

Z represents —(R⁵)ₙ—(Ar₂)ₘ, with n and m representing, independently of each other, 0 or 1, provided that at least one of n or m is 1, where
R⁵ represents, a linear, branched or cyclic, saturated or unsaturated, C₁-C₆ alkyl or alkylamido group, optionally comprising one or more heteroatom(s) chosen among O, N or S, and being optionally substituted with one or more halogen group(s), and
Ar₂ represents a C₆-C₁₀ aryl group substituted with at least one R as above-defined,
   as proteasome activity modulator in the manufacture of a pharmaceutical composition intended to prevent and/or treat a disease condition mediated by the proteasome activity.

The invention also relates to isoform of compounds of formula (I).

Within the meaning of the invention, the term “isoform” is intended to mean tautomers, stereoisomers, polymorphous forms or pharmaceutically acceptable solvates.

The term “tautomer” is intended to mean isomers, the structure of which differ by the position of one atom, typically one hydrogen atom, and one or more multiple bonds and which are able to easily and reversibly transform into each other.

The term “stereoisomer” is intended to mean isomers from a molecule which are identical in constitution but which differ only by one or more different arrangements of their atoms in space.

The terms “polymorphous form” are intended to mean compounds obtained by crystallization of a compound of general formula (I) in different conditions, as for example the use of different sequences, usually used for crystallization. Crystallization at different temperature implies, for example, various mode of cooling, such as very fast to very low cooling, or warming or melting steps of compounds followed by fast or gradual cooling.

The presence of polymorphous forms may be identified by NMR spectroscopy, IR-spectroscopy (infrared), differential scanning calorimetry (DSC), X-ray diffraction or other similar techniques known in the art.

Within the meaning of the invention, the term “unsaturated” with respect to a group from the formula (I) is intended to mean that this group may comprise one or more multiple bond(s), such as double or triple bond(s).

When a given group from formula (I) comprises more than one unsaturated bonds, for example at least two double bonds, those unsaturated bonds may or may not be conjugated between them in said group and/or conjugated with unsaturated bond(s) of the other moieties of the formula (I).

In particular, with respect to alkyl group, unsaturated is intended to mean alkenyl or alkenyl group.

The expression “branched cyclic alkyl group” intends to encompass cycloalkyl groups bearing alkyl substituent(s) as well as alkyl(cycloalkyl) groups optionally bearing alkyl substituent(s). Those hydrocarbon chains may be interrupted by one or more heteroatom(s) such as N, O or S.

With the meaning of the invention, the term “radical” with respect to Het is intended to mean that within the compounds of general formula (I) of the invention, the triazole or the oxadiazole group is covalently bonded with B and Z, and optionally further substituted with one or more, linear or branched, saturated or unsaturated, C₁-C₄ alkyl group.

According to one embodiment, Het may be substituted with one or more methyl or ethyl group, in particular with one methyl group. According to one embodiment, Het may be chosen from an 1,2,4-oxadiazole, an 1,3,4-oxadiazole, an 1,2,5-oxadiazole, an 1,2,3-oxadiazole, an 1,2,3-triazole, an 1,2,4-triazole- or a 4-methyl 1,2,4-triazole radical.

Advantageously, Het may be chosen from an 1,2,4-oxadiazole, an 1,2,5-oxadiazole, an 1,2,3-oxadiazole, an 1,2,3-triazole, an 1,2,4-triazole or a 4-methyl 1,2,4-triazole radical.

According to one embodiment, Het may be advantageously an 1,2,4-oxadiazole radical, an 1,3,4-oxadiazole radical or a 4-methyl 1,2,4-triazole radical.

Advantageously, Het may be an 1,2,4-oxadiazole radical or a 4-methyl 1,2,4-triazole radical.

According to one embodiment, Z may represent —(R⁵)ₙ—(Ar₂)ₘ.

According to one embodiment, n=1 and m=0.

According to another embodiment, n=0 and m=1.

According to another embodiment, n and m are 1.

According to one embodiment, R⁵ may be chosen from a linear, branched or cyclic, saturated or unsaturated, C₁-C₅ or preferably C₂-C₄ alkyl or alkylamido group, optionally comprising one or more heteroatom chosen from N, O or S.

According to one embodiment, R⁵ may be an alkylthioether, an N,N-aminoalkyl or an alkylether, with said alkyl group being as above-described.

According to another embodiment, R⁵ may be D-(CH₂)₁₋₄—C(O)N—, with D being S, O or N, and in particular R⁵ may be —S—CH₂—C(O)N—.

According to another embodiment, R⁵ may be chosen from a methyl, an ethyl, a propyl, an iso-propyl, a butyl, a sec-, a tert- or an iso-butyl group.

According to one embodiment, R⁵ may be chosen from a chloromethyl or a chloroethyl, and in particular is a chloromethyl.

Optionally, when m=0, R⁵ may further comprise at its free terminus one or more heteroatom(s) as above-indicated or one or more halogen group(s).

According to another embodiment, R⁵ may be chosen from a linear, branched or cyclic, saturated C₅-C₆ alkyl group comprising one or more heteroatom(s) chosen among O, N or S, and in particular chosen among N and O, and preferably may represent a N-morpholinylmethyl group, a N,N-diethylaminomethyl group or a N-piperidinylmethyl group.

In another embodiment, a nitrogen heterocycle derivative of the invention may be an oxadiazole derivative of formula (IIA) or (IIB):

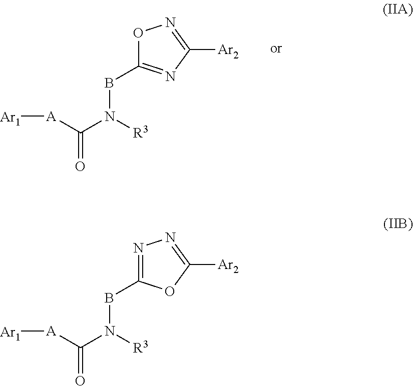

wherein Ar₁, Ar₂, A, B and R³ are as above-defined.

Advantageously, a nitrogen heterocyle derivative of the invention is an oxadiazole derivative of formula (IIA) as above-defined.

According to one embodiment, Ar₁ and Ar₂ may represent, independently of each other, a phenyl group or a napthyl group substituted with at least one R group.

More particularly, Ar₁ and Ar₂ may represent a phenyl group substituted with at least one R group.

According to another embodiment, the R group may be chosen among:

H, or

an halogen group, and in particular among Cl or Br, or

a linear, branched or cyclic, saturated or unsaturated, C₁-C₄ alkyl group.

In particular, R may be chosen from a methyl, an ethyl, a propyl, an isopropyl, a n-butyl, a sec-, a tert- or an iso-butyl group,

a linear, branched or cyclic, saturated or unsaturated, C₁-C₄ alkoxy group.

In particular, R may be chosen from a methoxy, an ethoxy, a propoxy or an iso-propoxy group, a n-butoxy, an iso-, a sec- or a tert-butoxy group.

a phenoxy group.

According to another embodiment, Ar₁ may be substituted with at least two R¹ groups, identical or different, said R¹ groups being as the above-defined R group.

According to another embodiment, R¹ may be chosen among:
H, or
Cl or Br, or
a methyl, an ethyl, a propyl, an iso-propyl, a n-butyl or an iso-, a sec- or a tert-butyl group, and in particular is a methyl, an ethyl or an iso-propyl group, or
a methoxy, an ethoxy, a propoxy or an iso-propoxy group, a n-butoxy or an iso-, a sec-, or a tert-butoxy group and in particular is a methoxy group.

According to one embodiment, Ar₂ may be a phenyl group substituted with at least one R group as above-defined.

According to another embodiment, Ar₂ may be substituted with at least two R² groups, identical or different, said R² groups being as the above-defined R group.

According to another embodiment, R² may be chosen among:
H, or
a methyl, an ethyl, a propyl, an iso-propyl, a n-butyl, an iso-, a sec- or a tert-butyl group, and in particular is a methyl group, or
a methoxy, an ethoxy, a propoxy or an iso-propoxy group, a n-butoxy, an iso-, a sec- or a tert-butoxy group, and in particular is a methoxy group.

According to one embodiment, R³ may represent H or a linear branched or cyclic, saturated or unsaturated, C₂-C₄ alkyl group and in particular may be chosen among a methyl, an ethyl, a propyl, an iso-propyl, a n-butyl, an iso-, a sec- or a tert-butyl, a vinyl, or an allyl group and in particular is an iso-propyl group or an allyl group.

According to one embodiment, A may represent a covalent bond or *-X—C(R⁴R⁶)-□, where *- and -□ are as above-defined, and X may represent a linear or branched, saturated or unsaturated, C₂-C₄ alkylene group or an heteroatom, and R⁴, and R⁶, independently of each other, may be chosen among H or linear or branched or cyclic, saturated or unsaturated, C₂-C₄ alkyl group.

According to one embodiment, X may be a methylene, an ethylene or a propylene group, and in particular may be a methylene group.

According to one embodiment, X may be a heteroatom chosen among O or N, and in particular may be O.

According to another embodiment, R⁴ and R⁶ may be, independently of each other, H or chosen among a methyl, an ethyl or a propyl group, and in particular may be a methyl group.

According to one embodiment, A may represent *-X—C(R⁴R⁶)-□, with *- and -□ being as above-defined, X being a methylene or O, and R⁴ and R⁶ being, independently of each other, H or a methyl group.

According to one embodiment, B may represent a linear, branched or cyclic, saturated or unsaturated, C₂-C₄ alkylene group, optionally substituted with one or more C₂-C₄ hydroxyalkyl group(s), or a phenylene or a naphtylene group.

According to another embodiment, B may represent a group chosen among a methylene, an hydroxymethylmethylene, an ethylene, a propylene, an iso-propylene, a phenylene or a naphtylene group, and in particular may represent a methylene, an hydroxymethylmethylene or a phenylene group.

According to another embodiment, a nitrogen heterocycle derivative of the invention may be an oxadiazole derivative of formula (IIIA):

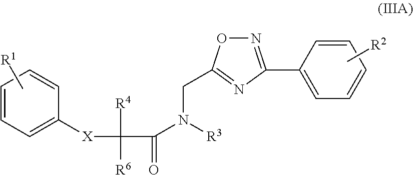

wherein

R¹, R², R³, R⁴ and R⁶ are as above-defined.

According to one embodiment, R¹ is in a position para and/or meta with respect to the group or heteroatom figured by X.

According to one embodiment, R¹ is in a position para with respect to X.

According to one embodiment, R² is/are in a position para and/or meta with respect to the oxadiazole radical.

According to another embodiment, when R² is an alkyl group as above-defined, R² is preferably in position meta with respect to the oxadiazole radical.

According to one embodiment, when R² is an alkoxy group as above-defined or a phenoxy group, R² is in position para with respect to the oxadiazole radical.

According to one embodiment, a nitrogen heterocycle derivative of the invention may be an oxadiazole of formula (IIIA) as above-defined, wherein R¹, R², R³, R⁴ and R⁶ are as defined in the following table (I):

| R¹ | | R² | R³ | R⁴, R⁶ | X |
|---|---|---|---|---|---|
| 1 | p-CH(CH₃)₂ | p-CH₃ | —CH(CH₃)₂ | H | O |
| 2 | p-CH(CH₃)₂ | m-CH₃ | —CH(CH₃)₂ | H | O |
| 3 | p-CH₂CH₃ | m-CH₃ | —CH(CH₃)₂ | H | O |
| 4 | p-Br | m-CH₃ | —CH(CH₃)₂ | H | O |
| 5 | p-O—CH₃ | m-CH₃ | —CH(CH₃)₂ | H | O |
| 6 | p-O—CH₃ | H | —CH(CH₃)₂ | H | O |
| 7 | p-CH₂—CH₃ | p-O—CH₃ | —CH(CH₃)₂ | H | O |
| 8 | H | p-O—CH₃ | —CH₂—CH═CH₂ | H | O |
| 9 | H | p-O—CH₃ | —CH(CH₃)₂ | H | O |
| 10 | p-CH₃ | p-O—CH₃ | —CH(CH₃)₂ | H | O |
| 11 | p-CH₃, o-Br | p-O—CH₃ | —CH(CH₃)₂ | H | O |
| 12 | p-Br | p-O—CH₃ | —CH(CH₃)₂ | H | O |
| 13 | m-CH₃ | p-O—CH₃ | —CH(CH₃)₂ | H | O |
| 14 | H | p-O—CH₃ | —CH(CH₃)₂ | H, | O |
| | | | | —CH₃ | |
| 15 | m-CH₃ | m-CH₃ | —CH(CH₃)₂ | H | O |
| 16 | p-O—CH₃ | p-CH₃ | —CH(CH₃)₂ | H | O |
| 17 | H | p-O—CH₃ | —CH(CH₃)₂ | H | —CH₂ |
| 18 | p-CH₃ | H | —CH₂—CH═CH₂ | H | O |
| 24 | p-Br | p-O—Ph | —CH(CH₃)₂ | H | O |
| 25 | p-Br | p-O—CH₃ | —CH(CH₃)₂ | H | O |
| | | m-O—CH₃ | | | |

or of formula (IIIB)

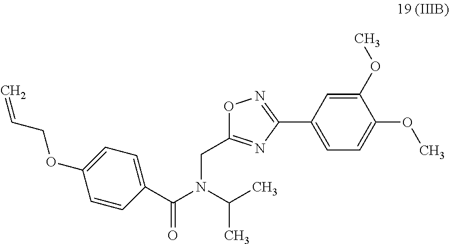

or of formula (IIIC)

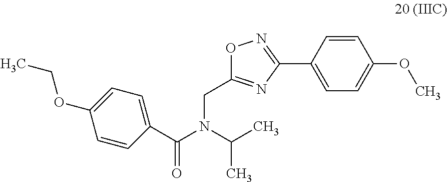

or of formula (IIID)

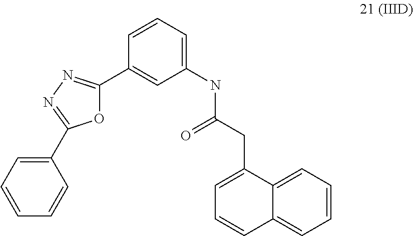

or of formula (IIIE)

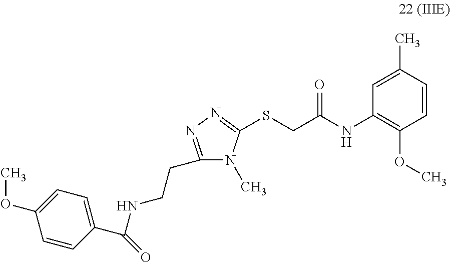

or of formula (IIIF)

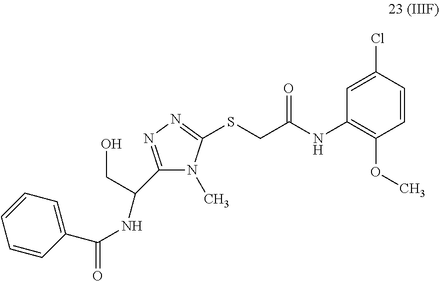

According to one embodiment, a nitrogen heterocycle derivative of the invention may be an oxadiazole derivative advantageously selected from compounds 3, 4, 7 and 12, as above-defined.

According to another embodiment, a nitrogen heterocycle derivative of the invention may be an oxadiazole derivative of formula (IV):

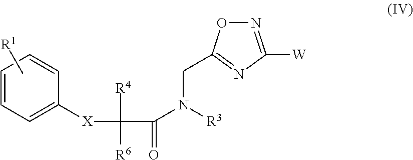

wherein

R₁, R₃, R₄, R₆ and X may be as above-defined, and

W may be chosen among a linear, branched or cyclic, saturated or unsaturated, C₁-C₆ or C₁-C₅ alkyl or alkylamido group, optionally comprising one or more heteroatom(s) chosen among O, N or S, and optionally substituted with one or more halogen group(s), or a C₆-C₁₀ (alkyl)aryl group, and preferably may be chosen among a linear or branched C₁-C₅ alkyl group or a C₆-C₁₀ (alkyl)aryl group and in particular a benzyl group.

In particular, W may be chosen from a methyl, an ethyl, a propyl, an iso-propyl, a butyl, a sec-, a tert- or an iso-butyl group, said group being optionally substituted with one or more halogen group(s), in particular with one chlorine atom, or from a naphtyl group or a benzyl group, and preferably may represent a methyl, a chloromethyl, an iso-propyl, a tert-butyl group, a benzyl group or a naphtyl group.

According to another embodiment, W may be chosen from a linear, branched or cyclic saturated C₅-C₆ alkyl group comprising one or more heteroatom(s) chosen among O and N, and preferably may represent a N-morpholinylmethyl group, a N,N-diethylaminomethyl group or a N-piperidinylmethyl group.

According to one embodiment, R₁ may represent a halogen group, and in particular may be Br.

According to another embodiment, X may be a heteroatom chosen among O or N, and in particular may represent O.

According to another embodiment, R₄ and R₆ may be, independently of each other, H or a methyl group, and in particular may both represent H.

According to another embodiment, R₃ may represent H or a linear or branched C₂-C4 alkyl group, and in particular may be an iso-propyl group.

According to one preferred embodiment, R₁ is in a position para with respect to X.

According to another embodiment, a nitrogen heterocycle derivative of the invention may be an oxadiazole derivative of formula (IV) as above defined, wherein

R₁ may represent a halogen group, in particular may be Br,

R₃ may represent H or a linear or branched C₂-C₄ alkyl group, and in particular may be an iso-propyl group,

R₄ and R₆ may be, independently of each other, H or a methyl group, and in particular may both represent H,

X may be a heteroatom chosen among O or N, and in particular may represent O, and

W is chosen among a linear or branched C₁-C₆ or C₁-C₅ alkyl group optionally substituted with one or more halogen group(s) in particular a chlorine atom, or from a naphtyl group or a benzyl group, and in particular is chosen from a methyl, a chloromethyl, an ethyl, a propyl, an iso-propyl, a butyl, a sec-, a tert- or an iso-butyl group, a naphtyl group or a benzyl group, and preferably may represents a methyl, a chloromethyl, an iso-propyl, a tert-butyl group, a benzyl group or a naphtyl group.

According to another embodiment, a nitrogen heterocycle derivative of the invention may be an oxadiazole derivative of formula (IV) as above defined, wherein W represents a methyl, chloromethyl, iso-propyl, tert-butyl, naphtyl group or benzyl group; R₁ is Br; X is O; R₄ and R₆ are both H; and R₃ is an iso-propyl group.

Such compounds of formula (IV) may be obtained according to a method of preparation as exemplified in Example 4.

According to another embodiment, a nitrogen heterocycle derivative of the invention may be an oxadiazole derivative of formula (IV) as above defined, wherein

R₁ may represent a halogen group, in particular may be Br,

R₃ may represent H or a linear or branched C₂-C₄ alkyl group, and in particular may be an iso-propyl group,

R₄ and R₆ may be, independently of each other, H or a methyl group, and in particular may both represent H,

X may be a heteroatom chosen among O or N, and in particular may represent O, and

W is chosen among a linear, branched or cyclic saturated C₅-C₆ alkyl group comprising one or more heteroatom(s) chosen among O and N, and preferably may represent a N-morpholinylmethyl group, a N,N-diethylaminomethyl group or a N-piperidinylmethyl group.

According to another embodiment, a nitrogen heterocycle derivative of the invention may be an oxadiazole derivative of formula (IV) as above defined, wherein W represents a N-morpholinylmethyl group, a N,N-diethylaminomethyl group or a N-piperidinylmethyl group; R₁ is Br; X is O; R₄ and R₆ are both H; and R₃ is an iso-propyl group.

According to one embodiment, a nitrogen heterocycle derivative of the invention may be an oxadiazole of formula (IV) as above-defined, wherein R¹, R³, R⁴, R⁶ and W are as defined in the following table (II):

| R¹ | R³ | R⁴, R⁶ | X | W |
|---|---|---|---|---|
| 27 p-Br | —CH(CH₃)₂ | H | O | —CH₃ |
| 28 p-Br | —CH(CH₃)₂ | H | O | —CH(CH₃)₂ |
| 29 p-Br | —CH(CH₃)₂ | H | O | —C(CH₃)₃ |
| 30 p-Br | —CH(CH₃)₂ | H | O | —CH₂-Ph |
| 31 p-Br | —CH(CH₃)₂ | H | O | —CH₂-Cl |
| 32 p-Br | —CH(CH₃)₂ | H | | 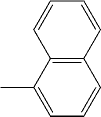 |
| 33 p-Br | —CH(CH₃)₂ | H | O | 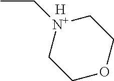 |
| 34 p-Br | —CH(CH₃)₂ | H | O | 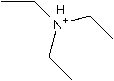 |
| 35 p-Br | —CH(CH₃)₂ | H | O | 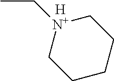 |

As exposed in the examples section, a nitrogen heterocycle derivative of the invention, in particular an oxadiazole derivative of the invention, may present an IC₅₀ (concentration of compound able to half inhibit an enzymatic activity rate as compared to said enzymatic activity rate without said compound) on the chymotrypsin-like activity lower or equal to 150 μM, in particular lower or equal to 85, in particular lower or equal to 80 μM, in particular lower or equal to 50 μM, in particular lower or equal to 20, in particular lower or equal to 10 μM, in particular lower or equal to 5 μM, in particular lower or equal to 2 μM, in particular lower or equal to 1 μM, in particular lower or equal to 0.6 μM, in particular lower or equal to 0.2 μM and more particularly lower or equal to 0.1 μM.

Additionally, a nitrogen heterocycle derivative of the invention may be an oxadiazole derivative that exhibits an EC₅₀ (concentration of a compound able to induce half the effect of a given pharmacological effect as compared to the maximum effect obtained with said compound) with regard to their toxicity on tumoral cells, lower or equal to 10 μM, in particular lower or equal to 8 μM, in particular lower or equal to 5 μM, in particular lower or equal to 2 μM and more particularly lower or equal to 1 μM.

A nitrogen heterocycle derivative of the invention may present a low molecular weight, in particular lower than 600 g/mol, in particular lower than 550 g/mol, and more particularly lower than 500 g/mol, and have a non-peptide-like structure.

Pharmaceutical or Cosmetic Compositions

The term “pharmaceutical” or “medicament”, used herein interchangeably, refers to an agent or mixture of agents that is primarily intended to treat and/or ameliorate and/or prevent a disease or a disorder or to diagnostic a disease or a disorder.

The term “pharmaceutically acceptable” means that which is useful in preparing a pharmaceutical composition that is generally safe, non-toxic, and neither biologically nor otherwise undesirable and includes what is acceptable for veterinary as well as human pharmaceutical use.

Within the meaning of the invention, the term “cosmetic composition” is intended to have the meaning as exposed in the European Directive 76/768/CEE.

More particularly, a “cosmetic composition” may be any substance or preparation intended to be placed in contact with the various external parts of the human body (skin, hair, nail, lips, . . . ), or with the teeth or mucous membranes of the oral cavity for, exclusively or mainly, cleaning them, perfuming them, changing their appearance, and/or correcting body odors, and/or protecting them or keeping them in good condition.

According to one embodiment, a nitrogen heterocycle derivative, and in particular an oxadiazole or triazole derivative, of the invention may be used as a cosmetic agent.

An “effective amount” means an amount sufficient to induce a positive modification in the condition to be regulated or treated, but low enough to avoid serious side effects. An effective amount may vary with the cosmetic or pharmaceutical effect to obtain or with the particular condition being treated, the age and physical condition of the end user, the severity of the condition being treated/prevented, the duration of the treatment, the nature of other treatments, the specific compound or product/composition employed, the route of administration, and like factors.

The term “subject” or “individual”, used interchangeably herein, means mammals and non-mammals. Examples of mammals include, but are not limited to: humans; non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. Examples of non-mammals include, but are not limited to, birds, and the like. The term “subject” or “individual” does not denote a particular age or sex.

A nitrogen heterocycle derivative, in particular an oxadiazole or a triazole derivative, of the present invention may be administered in an effective amount by any of the accepted modes of administration in the art.

In one embodiment, a nitrogen heterocycle derivative of the invention, in particular an oxadiazole or a triazole derivative, may be used in a composition intended to be administered by oral, nasal, sublingual, aural, ophthalmic, topical, rectal, vaginal, urethral, or parenteral injection route.

The route of administration and the galenic formulation will be adapted by one skilled in the art pursuant to the desired cosmetic or pharmaceutical effect.

In one embodiment, suitable concentration may range from 0,0001 mg/kg/d to 50 mg/kg/d, in particular from 0.001 mg/kg/d to 5 mg/kg/d and more particularly from 0.01 to 0.5 mg/kg/d, depending upon numerous factors such as the age and relative health of the subject, the potency of the formulation used, and the therapeutic or cosmetic indication towards which the administration is directed.

One of ordinary skill in the art of therapeutic formulations or cosmetic formulations will be able, without undue experimentation and in reliance upon personal knowledge and the disclosure of this application, to ascertain a therapeutically or cosmetically effective amount of a nitrogen heterocycle derivative of the invention for a given indication.

A pharmaceutical composition of the invention may be formulated with any known suitable pharmaceutically acceptable carrier according to the dose, the galenic form, the route of administration and the likes.

As used herein, “pharmaceutically acceptable carrier” includes any and all solvents, dispersion media, coatings, antibacterial and antifungal agents, isotonic and absorption delaying agents and the like. Except insofar as any conventional media or agent is incompatible with the active ingredient, its use in a medicament of the invention is contemplated.

A pharmaceutical or a cosmetic composition of the invention may be in the form of tablets, pills, powders, lozenges, sachets, cachets, elixirs, suspensions, emulsions, solutions, syrups, aerosols, sprays, ointments, gels, creams, sticks, lotions, pastes, soft and hard gelatin capsules, suppositories, sterile injectable solutions, sterile packaged powders and the likes.

According to one embodiment, a cosmetic composition of the invention may be in particular adapted to be administered by topical route.

A cosmetic composition of the invention may comprise any excipient usually used in the cosmetic art, such as hydrophilic or lipophilic gelifying agent, hydrophilic or lipophilic additives, antioxidants, conservative agents, solvents, perfumes, fillers, UV screens, moisturizers, odor absorbing agents, and coloring agents.

According to one embodiment, a pharmaceutical composition of the invention may be intended to be administered separately, sequentially or simultaneously with an agent useful for the prevention and/or the treatment of a disease condition, in particular a cancer condition, said agent being different from the nitrogen heterocycle derivative of the invention.

According to one embodiment, a pharmaceutical composition of the invention may be intended to be administered separately, sequentially or simultaneously with a chemotherapeutic agent or a radiotherapeutic regimen.

Kit-of-Parts

The invention is also directed to a novel kit-of-parts that is suitable for use in the treatment of cancers.

A kit-of-part of the invention may comprise (i) a nitrogen heterocycle derivative, in particular an oxadiazole or a triazole derivative, as defined above, and (ii) at least one agent useful for the prevention and/or the treatment of a cancer condition, said agent being different of said nitrogen heterocycle derivative (i).

According to one embodiment, the kit-of-part of the invention may comprise (i) a nitrogen heterocycle derivative, in particular a triazole or an oxadiazole derivative, as defined above, and (ii) at least one agent as above-defined, each of (i) and (ii) being laid out to be administered separately, sequentially or simultaneously.

An agent useful for the prevention and/or the treatment of a cancer condition may be a chemotherapeutic agent or a radiotherapeutic agent.

As example of chemotherapeutic agents that may be suitable for the invention, one may mention chemotherapeutic agents chosen from alkylating agents, nitrosoureas, anti-metabolite agents, anti-tumor antibiotics, plant alkaloids, steroid hormones, monoclonal antibodies, and mixtures thereof.

As example of alkylating agents that may be used in accordance with the invention, one may mention chlorambucil and cyclophosphamide.

As example of nitrosoureas that may be used in accordance with the invention, one may mention carmustine and lomustine.

As example of anti-metabolite agents that may be used in accordance with the invention, one may mention fludarabine, 6-mercaptopurine and 5-fluorouracil (5 FU).

As example of anti-tumor antibiotics that may be used in accordance with the invention, one may mention the mitomycin-C, the bleomycin, and the anthracyclines such as the doxorubicine.

As example of plant alkaloids that may be used in accordance with the invention, one may mention vincristine and vinblastine.

As example of steroid hormones that may be used in accordance with the invention, one may mention tamoxiphen.

As example of monoclonal antibodies that may be used in accordance with the invention, one may mention rituximab and alemtuzumab.

According to one embodiment, a kit-of-parts the invention may comprise (i) at least one nitrogen heterocycle derivative, as defined above, and (ii) at least one agent useful for the prevention and/or the treatment of a cancer condition, said agent being different of said nitrogen heterocycle derivative and in particular being chosen among histone deacetylase inhibitors.

A histone deaceatylase inhibitor (HDAC inhibitors or HDI) are a class of compounds that interfere with the function of histone deacatylase.

Among the HDIs that may be used in the invention, one may mention hydroxamic acids, such as trichostatin 1, cyclic tetrapeptides, such as trapoxin B and the depsipeptides, the benzamides, the electrophilic ketones, and the aliphatic acids compounds such as phenylbutyrate and valproic acid.

According to another embodiment, a HDI that may be used in accordance with the invention may be, for example, SAHA/vorinostat, belinostat/PXD1001, MS275, LAQ824/LBH589, C1994, or MGCD0103.

According to one embodiment, the additional agent useful for the prevention and/or the treatment of a cancer condition (ii) may be an agent useful for the prevention and/or the treatment of B cell lymphoma, and more particularly for the prevention and/or treatment of multiple myeloma or mantle cells lymphoma.

As example of such suitable agent, one may mention melphalan, vincristine, doxorubicin, cyclophosphamide, fludarabine, thalidomide, prednisone or dexamethasone, cytosine arabinoside, methotrexate or rituximab.

Advantageously, those agents may be used in combination more particularly adapted to a given disease condition such as melphalan and prednisone or thalidomide and dexamethasone, or cyclophosphamide and fludarabine or vincristine and doxorubicin and dexamethasone for multiple myeloma or rituximab, cyclophosphamide, doxorubicin, vincristine and prednisone for mantle cells lymphoma.

As example of radiotherapeutic agent that may be used in accordance with the invention, one may mention an isotope such as for example chosen from ¹⁴C, ³H, or ¹²⁵I, ¹³¹I, ³²P, ⁸⁹Sr, ⁹⁰Y, administered as radio-labeled antibodies

Methods of Treatment

According to one embodiment, the instant invention relates to a method for preventing and/or treating a disease condition comprising at least a step of administering to an individual in need thereof at least an effective amount of at least one nitrogen heterocycle derivative, in particular at least one triazole or one oxadiazole derivative, in accordance with the invention.

The disease condition may be chosen among cancers, immunological diseases, auto-immune diseases, allograft rejections, viral diseases, such as mumps, measles, Rous sarcoma or AIDS, parasitic diseases such as malaria or trypanosome, bacterial infections, such as tuberculosis, inflammatory diseases, such as polyarthritis or liver inflammation, cardiac diseases and ischemic strokes, such as myocardial, cerebral or pulmonary ischemic injuries, muscular dystrophies, muscle wasting, traumatisms, burns, disease conditions associated with aging, such as neurodegenerative diseases.

According to one embodiment, a disease condition may be chosen among lung and oro-pharynx cancers, colo-rectal cancers, gastro-intestinal tract cancers, breast cancers, prostate cancers, pancreas cancers, leukemias such as Hodgkin's disease, immunoprofilerative tumors, in particular multiple myeloma, bladder cancers, kidney cancers, ovarian cancers, cervical cancers, brain cancers, head and neck cancers, skin cancers, in particular melanoma, bone cancers.

According to one embodiment, a disease condition may be a cancer condition, and in particular a B-cells lymphoma.

According to another embodiment, a disease condition may be chosen among follicular lymphoma, small non-cleaved cell lymphoma, such as endemic Burkitt's or sporadic Burkitt's or non-Burkitt's lymphoma, marginal zone lymphoma, such as mucosa-associated lymphoid tissue (MALT-oma) (extranodal), or monocytoid B-cell lymphoma (nodal) or splenic lymphoma with villous lymphocytes, mantle cell lymphoma, large cell lymphoma, such as diffused large cell, or diffused mixed cell or immune-blastic lymphoma or primary mediastinal B-cell lymphoma or angiocentric lymphoma-pulmonary B-cell, and small lymphocytic lymphoma.

According to another embodiment, a disease condition may be a mantle cell lymphoma.

According to another embodiment, a disease condition may be a multiple myeloma.

According to one embodiment, a method of the invention may comprise the step of administering a nitrogen heterocycle derivative of the invention, in particular an oxadiazole or a triazole derivative, in accordance with the invention separately, sequentially or simultaneously with a chemotherapeutic agent or a radiotherapeutic regimen.

A chemotherapeutic agent may be as above-described.

A radiotherapeutic regimen may be administered by exposing an individual in need thereof to a source of ionizing radiation such as X-ray, gamma-ray or beta-ray.

A source of ionizing radiation that may convene to the invention may be, for example external source such as radioactive cobalt or a digital linear accelerator producing X-rays or an administrated source under the form of an isotope such as for example ¹⁴C, ³H, or ¹²⁵I, ¹³¹I, ³²P, ⁸⁹Sr, ⁹⁰Y.

For example, the isotopes may be administered as radio-labeled antibodies.

According to another embodiment, the invention is directed to a use of at least one nitrogen heterocycle derivative of the invention, in particular an oxadiazole or triazole derivative in accordance with the invention, in a cosmetic composition for the prevention and/or the treatment of skin aging.

In such an embodiment, a compound of the invention that may convene may be a proteasome activator.

The skin aging may be from chronologic origin, and/or may be indicative of a cutaneous condition, resulting, for example, from the photoaging.

Accordingly, a cosmetic composition of the invention may be intended to prevent and/or treat a thinning of an epidermis and/or a lost of firmness, elasticity, density and/or tonicity of an epidermis and/or the formation of wrinkles.

According to another embodiment, the invention relates to a cosmetic method for preventing and/or treating the skin aging comprising at least the step of administering to an individual in need thereof at least one effective amount of a nitrogen heterocycle derivative of the invention, in particular an oxadiazole or triazole derivative of the invention.

The present invention will be better understood by referring to the following examples which are provided for illustrative purpose only and should not be interpreted as limiting in any manner the instant invention.

### BRIEF DESCRIPTION OF THE DRAWINGS

FIG. 1: Percentage of inhibition of the CT-L active site by increasing concentration of the compound 13 at pH 8 and 37° C.

FIG. 2: Cytotoxic effect of the compound 2 on HeLa cells treated for 48 h at 37° C. The cell viability is determined with an XTT assay.

### EXAMPLES

### Example 1

Effects on Proteasome Activities

The 23 following compounds (1-23) were known and commercially available from ChemBridge corporation (www.chembridge.com). Compounds 24, 25 and 27-35 were synthesized. The compounds were dissolved in DMSO to 10 mM stock concentrations and stored at −20° C.

Rabbit erythrocyte 20S proteasome was obtained from Boston Biochem, Cambridge, USA. The fluorogenic substrates Suc-LLVY-AMC, Boc-LRR-AMC and Z-LLE-βNA used to measure the proteasome activities CT-L, T-L and PA respectively were purchased from Bachem (France). Other reagents and solvents were purchased from commercial sources. Fluorescence was measured using a BMG Fluostar microplate reader.

Compounds 1-23 were tested for their potential to inhibit the CT-L, T-L and PA activities of the rabbit reticulocyte 20S proteasome. Enzyme activities were determined by monitoring the hydrolysis of the appropriate fluorogenic substrate (λ_{exc}=360, λₑₘ=465 nm for AMC substrates, and λ_{exc}=340, λₑₘ=405 nm for the βNA substrate) for 1 h at 37° C.

In the in vitro screening, each compound was tested in duplicate against the three activities, at 100 μM and 50 μM in the corresponding buffer. The buffer were (pH 8): 50 mM Tris, 150 mM NaCl, 10% (v/v) glycerol, 0.025% (w/v) SDS, and 3% (v/v) DMSO (CT-L and PA activities); 50 mM Tris, 150 mM NaCl, 10% (v/v) glycerol, and 3% (v/v) DMSO (T-L activity). Compounds with inhibitor efficiency superior at 50% at 100 μM for any proteasome activity were retested with 0.1-100 μM of test compound.

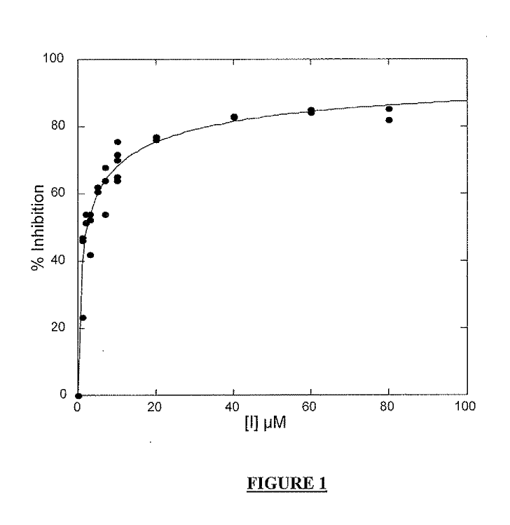

The inhibitory activity of compounds is expressed as IC₅₀, which corresponds to the concentration of proteasome inhibitor leading to a loss of activity of 50%. The values of IC₅₀ were calculated by fitting the experimental data to equation 1: % Inhibition=100×(1−Vᵢ/V₀)=100 [I]₀/(IC₅₀+[I]₀), or equation 2: % Inhibition=100 [I]₀^{nH}/(IC₅₀^{nH}+[I]₀^{nH}), where Vᵢ is the initial rate in the presence of the inhibitor, V₀ is the initial rate in the absence of the inhibitor, [I]₀ is the inhibitor concentration, nH is the Hill number (FIG. 1).

FIG. 1 is illustrative of an inhibition curve obtained with compound 13 with respect to the CT-L activity.

Compounds 24, 25 and 31-35 were tested in duplicate against three activities of human erythrocyte 20S proteasome at 100 and 50 μM in the appropriate buffer (pH 7.5): 20 mM Tris HCl, 10% (v/v) glycerol, 0.01% (w/v) SDS and 2% DMSO (CT-L and PA activities); 20 mM Tris HCl, 10% (v/v) glycerol, and 2% DMSO (T-L activity). The inhibitory activities were analyzed as described above for compounds 1-23.

The following table III summarizes the results obtained for a selected series of oxadiazole derivatives.

TABLE III

| | | | | | IC₅₀ (μM) or | | |
|---|---|---|---|---|---|---|---|
| | | | | | % inhibition | IC₅₀ (μM) or | IC₅₀ (μM) or % |
| | | | | | (inhibitor | % inhibition | inhibition |
| | | | | | concentration) | (inhibitor | (inhibitor |
| | | | | | on | concentration) | concentration) |
| | | | | | chymotrypsin- | on post-acid | on trypsin-like |
| Structure | | | | | like activity | activity | activity |
| 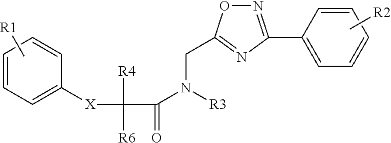 | | | | | | | |
| R1 | R2 | R3 | R4, R6 | X | | | |
| 1 p-CH(CH₃)₂ | p-CH₃ | —CH(CH₃)₂ | H | O | ≈7 | NI | NI |
| 2 p-CH(CH₃)₂ | m-CH₃ | —CH(CH₃)₂ | H | O | ≈1 | NI | NI |
| 3 p-CH₂CH₃ | m-CH₃ | —CH(CH₃)₂ | H | O | 0.17 ± 0.01 | NI | NI |
| 4 p-Br | m-CH₃ | —CH(CH₃)₂ | H | O | 0.51 ± 0.07 | NI | 7.3% |
| | | | | | | | (50 μM) |
| 5 p-O—CH₃ | m-CH₃ | —CH(CH₃)₂ | H | O | 0.91 ± 0.12 | NI | NI |
| 6 p-O—CH₃ | H | —CH(CH₃)₂ | H | O | 1.02 ± 0.16 | NI | ND |
| 7 p-CH₂—CH₃ | p-O—CH₃ | —CH(CH₃)₂ | H | O | 0.090 ± 0.006 | NI | NI |
| 8 H | p-O—CH₃ | —CH₂— | H | O | 1.66 ± 0.12 | NI | NI |
| | | CH═CH₂ | | | | | |
| 9 H | p-O—CH₃ | —CH(CH₃)₂ | H | O | 3.96 ± 0.75 | NI | NI |
| 10 p-CH₃ | p-O—CH₃ | —CH(CH₃)₂ | H | O | 0.71 ± 0.04 | NI | NI |
| 11 p-CH₃, | p-O—CH₃ | —CH(CH₃)₂ | H | O | 50% (50 | NI | NI |
| o-Br | | | | | μM) | | |
| 12 p-Br | p-O—CH₃ | —CH(CH₃)₂ | H | O | 0.10 ± 0.01 | NI | NI |
| 13 m-CH₃ | p-O—CH₃ | —CH(CH₃)₂ | H | O | 2.33 ± 0.28 | NI | 28% (100 μM) |
| 14 H | p-O—CH₃ | —CH(CH₃)₂ | H, | O | 8.05 ± 0.83 | NI | NI |
| | | | —CH₃ | | | | |
| 15 m-CH₃ | m-CH₃ | —CH(CH₃)₂ | H | O | ≈27 | NI | NI |
| 16 p-O—CH₃ | p-CH₃ | —CH(CH₃)₂ | H | O | 2.49 ± 0.91 | NI | NI |
| 17 H | p-O—CH₃ | —CH(CH₃)₂ | H | —CH₂ | 54% (50 μM) | NI | 9.5% (50 μM) |
| 18 p-CH₃ | H | —CH₂— | H | O | 0.33 ± 0.03 | NI | NI |
| | | CH═CH₂ | | | | | |
| 24 p-Br | p-O—Ph | —CH(CH₃)₂ | H | O | 0.61 ± 0.03 | NI | 10.8 ± 0.4 |
| 25 p-Br | p-O—CH₃ | —CH(CH₃)₂ | H | O | 0.037 ± 0.003 | NI | NI |
| | m-O—CH₃ | | | | | | |
| 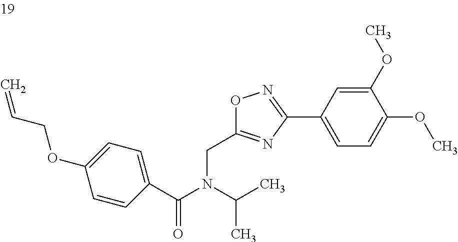 | | | | | 3.51 ± 0.12 | NI | Activation × 11.5 (100 μM) |
| 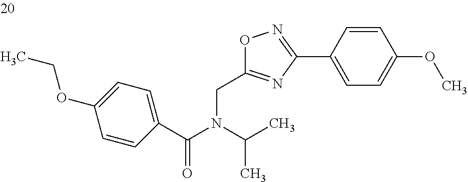 | | | | | ≈18 | NI | Activation × 3.4 (50 μM) |
| 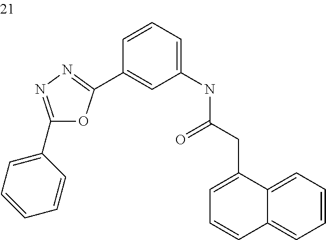 | | | | | 144 ± 22 | 22% (100 μM) | NI |
| 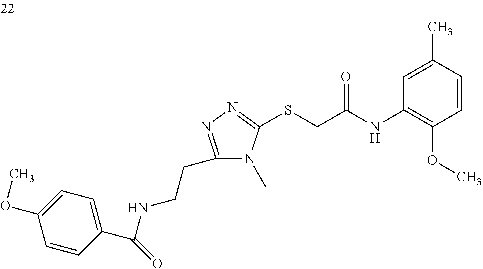 | | | | | 80 | 16% (100 μM) | 22% (100 μM) |
| 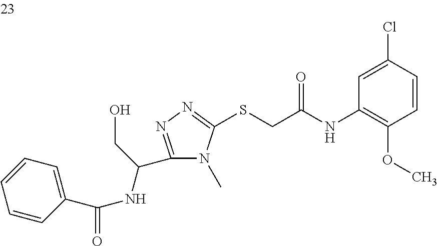 | | | | | 85 | NI | 50% (100 μM) |
| 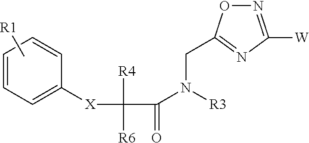 | | | | | | | |
| R1 | R3 | R4, R6 | X | W | | | |
| 27 p-Br | —CH(CH₃)₂ | H | O | —CH₃ | 12.3 ± 0.3 | 76 ± 2 | NI |
| 28 p-Br | —CH(CH₃)₂ | H | O | —CH(CH₃)₂ | 12.3 ± 0.3 | 45 ± 1 | NI |
| 29 p-Br | —CH(CH₃)₂ | H | O | —C(CH₃)₃ | 81 ± 4 | 28 ± 3 | NI |
| 30 p-Br | —CH(CH₃)₂ | H | O | —CH₂-Ph | 15 ± 1 | 54 ± 4 | NI |
| 31 p-Br | —CH(CH₃)₂ | H | O | —CH₂-Cl | 2.69 ± 0.22 | NI | NI |
| 32 p-Br | —CH(CH₃)₂ | H | O | 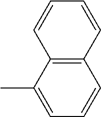 | 30% (100 μM) | NI | 29.1 ± 0.9 |
| 33 p-Br | —CH(CH₃)₂ | H | O | 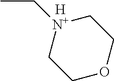 | 78.6 ± 1.6 | NI | 42% (100 μM) |
| 34 p-Br | —CH(CH₃)₂ | H | O | 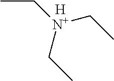 | 30.9 ± 2.3 | NI | 28% (100 μM) |
| 35 p-Br | —CH(CH₃)₂ | H | O | 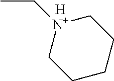 | 37.3 ± 4.2 | NI | 47% (100 μM) |

NI: No Inhibition

The reversible property of the inhibition was demonstrated by abolishing the inhibition in the presence of an excess of substrate.

The inhibitors act selectively on proteasome with no inhibition at 100 μM of human calpain-I (for example, compounds 6, 11, 16) and human cathepsin B (for example, compound 13), or very poor inhibition (for example, ≦30% inhibition of calpain-I at 100 μM for compounds 8 and 9).

### Example 2

Effects on Cell Viability

The cell survival was determined with the XTT assay. Human cells (HeLa from cervical carcinoma and HEK-293 from epithelial kidney) were obtained from Invitrogen (Cergy-Pontoise, France). The cells were grown at 37° C. in DMEM supplemented with 10% fetal bovine serum (Invitrogen) in a humidified atmosphere of 5% CO₂ and 95% air.

5×10³ cells in 100 μL culture medium were exposed for 48 h in 96-well plates to increasing concentrations of compounds: 5-100 μM, final concentration of DMSO is 1% (v/v). The culture medium is then replaced by 100 μL of DMEM F12 culture medium devoid of phenol red and containing a mixture of XTT (0.3 mg/mL) and PMS (8.3 nM) (XTT: 2,3-bis[2-methoxy-4-nitro-5-sulfophenyl-2H-tetrazolium-5-carboxyanilide sodium; PMS: phenazine methosulfate, both purchased from Sigma, Saint Quentin Fallavier, France). Cells were then incubated for 3 h at 37° C.

During this incubation, the mitochondrial deshydrogenases of viable cells hydrolyzed the tetrazolium cycle of XTT, leading to orange formazan crystals soluble in aqueous solution.

The orange color was measured at 485 nm using a BMG Fluostar microplate reader.

In control experiments, cells were treated by the vehicule (DMSO) at the same concentration than that used for the inhibitors.

The cytotoxicity activity of drags was expressed as the concentration inhibiting cell growth by 50% (EC₅₀) calculated from the survival curves. The experimental data are fitted to the following equation, describing the relationship between % survival and drug concentration (C):

% cell survival=100−(Eₘₐₓ×Cⁿ)/(Cⁿ+EC₅₀ⁿ)

where C is the drug concentration; Eₘₐₓ is the maximum drug effect (%); EC₅₀ is the concentration that produces one-half of the maximum effect; and n is the Hill constant, which describes the shape of the curve.

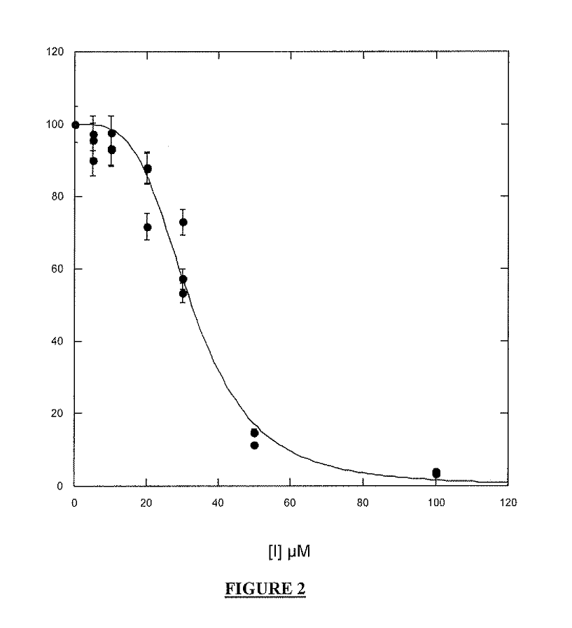

For example, EC₅₀=32±3.6 μM for compound 2 (FIG. 2) and 44±3.8 μM for compound 1 on HeLa cells; EC₅₀ of 39.2±2.0 μM for compound 19, 26.0±2.3 μM for compound 21, 16.1±0.5 μM for compound 31 on HEK-293 cells.

### Example 3

Inhibition of Proteasome in a Cell Assay

In order to demonstrate that the cytotoxic effect observed on tumor cells results from the specific inhibition of cellular proteasome, the accumulation of ubiquitinalyted proteins was determined.

Indeed, if the proteasomes are inhibited, in particular the proteasome 26S, the degradation of ubiquitinalyted proteins should be blocked, and those proteins should accumulate.

240 000 HEK-293 cells were incubated for 16 h, in presence of oxadiazole derivatives at the concentration of 10 μM.

After extraction of the proteins (centrifugation of lysed cells), the concentration of proteins in the sample was determined with the BCA (Bicinchoninic Acid) protein Assay.

3 μg of proteins for each sample were deposited into a SDS-PAGE gel at 8%.

The Western blot with an anti-ubiquitin antibody shows an accumulation of ubiquitinalyted proteins.

The oxadiazole derivatives are observed, as MG132 (aldehyde inhibitor of the proteasome) to induce an accumulation of ubiquitinalyted proteins.

Therefore, the proteasome inhibitors of the invention effectively cross the cell membranes and inhibit the proteasome.

### Example 4

Synthesis of Nitrogen Heterocycle Derivatives

Eleven nitrogen heterocycle derivatives corresponding to general formula IIIA (compounds 24 and 25) and to general formula IV (compounds 27 to 35) have been synthesized, as follows.

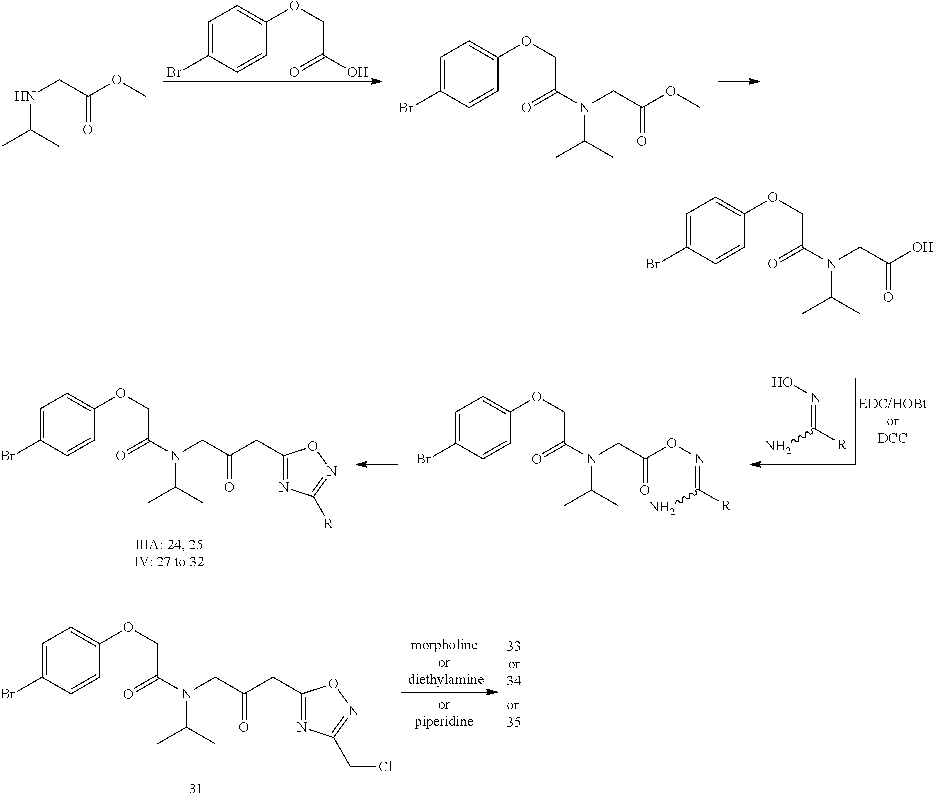

General Procedure for the Preparation of Compound 27 to 32 and 24-25

To a solution of isopropylamine (20 mL, 0.233 mol) in diethylether (100 mL) was added methyl bromoacetate (9.5 mL, 0.100 mol). The mixture was stirred for 68 h. and then treated by 50% aqueous sodium hydroxide (20 mL). The aqueous phase was extracted twice by ether. The combined organic phases were washed by water (20 mL), brine (20 mL), then dried over magnesium sulfate and concentrated in vacuo to afford methyl (isopropylamino)acetate (11.09 g, 85% yield).

Thionyl chloride (6.3 mL, 87 mmol) was added dropwise to 4-bromophenoxyacetic acid (3.47 g, 15 mmol). After refluxing for 4 h, the resulting mixture was concentrated in vacuo to afford the 4-bromophenoxyacetyl chloride (3.74 g, 15 mmol) which was used without further purification.

To a solution of 4-bromophenoxyacetic acid (3.46 g, 0.050 mol) in THF (50 mL) was added at 0° C., hydroxybenzotriazole (2.77 g, 0.018 mol), 1-ethyl-3-(3-dimethylaminopropyl)carbodiimide (EDC) (3.20 mL, 0.018 mol) and methyl(isopropylamino)acetate (2.16 g, 0,0165 mol). After stirring overnight at room temperature, the resulting mixture was concentrated in vacuo, diluted by water (100 mL) and extrated twice by methylene chloride. The resulting organic phase was washed by aqueous hydrochloric acid, water and dried over magnesium sulfate. After concentration in vacuo, methyl N-[2-(4-bromophenoxy)acetyl]-N-isopropyl glycinate was afforded (4.37 g, 85%).

Alternatively, to a solution of 4-bromophenoxyacetyl chloride (3.74 g, 15 mmol) in THF (10 mL) was added slowly triethylamine (2.1 mL, 15 mmol) and methyl (isopropylamino)acetate (2 g, 15.3 mmol). After stirring overnight at room temperature, the resulting mixture was concentrated in vacuo, diluted by aqueous 1M hydrochloric acid (15 mL) and extrated twice by methylene chloride (2*50 mL). The resulting organic phase was washed by aqueous 10% potassium hydrogenocarbonate and then by water, dried over sodium sulphate and concentrated in vacuo. The crude residue was purified by chromatography over silica gel (75% diethyl ether and 25% petroleum ether) to afford methyl N-[2-(4-bromophenoxy)acetyl]-N-isopropyl glycinate as a white solid (2.85 g, 55%) Mp:91° C. ¹H NMR (300 MHz, CDCl₃) δ (75/25 mixture of rotamers): 1.10 and 1.22 (d, J=6.9 Hz, 6H), 3.71 and 3.76 (s, 3H), 3.94 and 4.06 (s, 2H), 4.24 and 4.82 (hept, J=6.9 Hz, 1H), 4.63 and 4.76 (s, 2H), 6.85 (d, J=8.1 Hz, 2H), 7.38 (d, J=8.1 Hz, 2H). Anal. Calcd. for C₁₄H₁₈NO₄Br: C, 48.85; H, 5.27; N, 4.07. Found: C, 48.52; H, 5.29; N, 3.95.

A solution of methyl N-[2-(4-bromophenoxy)acetyl]-N-isopropyl glycinate (4.37 g, 12.7 mmol) in THF (15 mL) was reacted for 3 h with aqueous 1 M lithium hydroxide (20 mL, 20 mmol). The mixture was concentrated in vacuo and acidified to pH=1 with concentrated hydrochloric acid. After filtration, N-[2-(4-bromophenoxy)acetyl]-N-isopropyl glycine was afforded as a white solid (3.54 g, 84%). ¹H NMR (300 MHz, CDCl₃) δ (75/25 mixture of rotamers): 1.11 and 1.24 (d, J=6.6 Hz, 6H), 2.89 (s, 1H), 3.97 and 4.08 (s, 2H), 4.23 and 4.82 (hept, J=6.6 Hz, 1H), 4.63 and 4.77 (s, 2H), 6.83 (d, J=9.0 Hz, 2H), 7.38 (d, J=9.0 Hz, 2H).

To a solution of N-[2-(4-bromophenoxy)acetyl]-N-isopropyl glycine (0.330 g, 1.00 mol) in THF (4 mL) is added at 0° C., hydroxybenzotriazole (0.187 g, 1.22 mmol), EDC (0.22 mL, 1.24 mmol) and ethanamidoxime (84.7 mg, 1.14 mmol, prepared according to S. Org. Chem. 2003, 7316-7321). After stirring for two days at room temperature, the resulting mixture was concentrated in vacuo, diluted by water (20 mL) and extracted three times by methylene chloride. The organic phase was dried over magnesium sulfate, concentrated in vacuo and then purified by chromatography over silica gel (9 g, eluant petroleum ether and ethyl acetate) to afford the corresponding ester (0.149 g, 39%), which was refluxed for 4 h in toluene (10 mL). After concentration in vacuo and chromatography over silica gel (eluant 1% methanol in methylene chloride) compound 27 was afforded (110 mg, 76%). NMR ¹H (300 MHz, CDCl₃) δ (75/25 mixture of rotamers): 1.11 and 1.26 (d, J=6.8 Hz, 6H), 2.35 and 2.38 (s, 3H), 4.31 and 4.77 (hept, J=6.8 Hz, 1H), 4.60 and 4.71 (s, 2H), 4.77 (s, 2H), 6.83 (d, J=9 Hz, 2H), 7.38 (d, J=9 Hz, 2H). NMR ¹³C (75 MHz, CDCl₃) δ (75/25 mixture of rotamers): 11.5 (CH₃), 19.6 and 21.1 (CH₃ iPr), 36.8 and 37.9 (CH₂), 46.6 and 48.6 (CH), 67.4 and 68.1 (CH₂), 114 (C), 116.4 (CH), 132.3 (CH), 156.9 (C), 167.2 and 167.6 (C), 175.8 (C). Anal. Calcd. for C₁₅H₁₈N₃O₃Br: C, 48.93; H, 4.93; N, 11.41. Found: C, 48.88; H, 4.94; N, 11.13.

Compound 28

Similarly, reactions from isobutyramidoxime (89 mg, 0.86 mmol), N-[2-(4-bromophenoxy)acetyl]-N-isopropyl glycine (232 mg, 0.703 mmol) afforded compound 28 (85 mg, overall yield 30%). NMR ¹H (300 MHz, CDCl₃) δ (70/30 mixture of rotamers): 1.10 and 1.25 (d, J=6.8 Hz, 6H), 1.29 and 1.32 (d, J=7 Hz, 6H), 3.04 (m, 1H), 4.31 and 4.78 (hept, J=6.8 Hz, 1H), 4.62 and 4.70 (s, 2H), 4.77 and 4.81 (s, 2H), 6.84 (m, 2H), 7.38 (m, 2H). NMR ¹³C (75 MHz, CDCl₃) δ (70/30 mixture of rotamers): 19.6 and 20.3 (CH₃ NiPr), 21.1 and 22.6 (CH₃ iPr), 36.8 and 37.9 (CH₂), 46.6 and 48.6 (CH NiPr), 67.5 and 67.9 (CH₂), 113.9 (C), 116.4 (CH), 132.4 (CH), 157 (C), 167.5 (C), 175.0 (C), 175.6 (C). Anal. Calcd. for C₁₇H₂₂N₃O₃Br: C, 51.53; H, 5.60; N, 10.60. Found: C, 51.38; H, 5.61; N, 10.33.

Compound 29

Similarly, reactions from pivalamidoxime (279 mg, 2.4 mmol), N-[2-(4-bromophenoxy)acetyl]-N-isopropyl glycine (660 mg, 2.00 mmol) afford compound 29 (124 mg, overall yield 15%). NMR ¹H (300 MHz, CDCl₃) δ (70/30 mixture of rotamers): 1.10 and 1.25 (d, J=6.8 Hz, 6H), 1.33 and 1.35 (s, 9H), 4.31 and 4.78 (hept, J=6.8 Hz, 1H), 4.63 and 4.68 (s, 2H), 4.77 and 4.82 (s, 2H), 6.83 (d, J=9 Hz, 2H), 7.38 (d, J=9 Hz, 2H). NMR ¹³C (75 MHz, CDCl₃) δ (70/30 mixture of rotamers): 19.6 and 21.1 (CH₃ iPr), 22.6 (CH₃), 28.2 (C), 36.8 and 37.9 (CH₂), 46.6 and 48.5 (CH), 67.5 and 67.8 (CH₂), 113.9 (C), 116.4 (CH), 132.4 (CH), 156.9 and 157.0 (C), 167.5 (C), 175.4 and 176.6 (C), 177.6 (C). Anal. Calcd. for C₁₈H₂₄N₃O₃Br: C, 52.60; H, 5.90; N, 10.24. Found: C, 52.58; H, 5.95; N, 10.00.

Compound 30

Similarly, reactions from benzylamidoxime (249 mg, 1.64 mmol), N-{2-(4-bromophenoxy)acetyl]-N-isopropyl glycine (452 mg, 1.36 mmol) afford compound 30 (107 mg, overall yield 20%). NMR ¹H (300 MHz, CDCl₃) δ (75/25 mixture of rotamers): 1.09 and 1.23 (d, J=6.8 Hz, 6H), 4.04 (s, 2H), 4.30 and 4.77 (hept, J=6.8 Hz, 1H), 4.61 and 4.69 (s, 2H), 4.74 and 4.76 (s, 2H), 6.73 and 6.83 (d, J=9 Hz, 2H), 7.25-7.38 (m, 7H). NMR ¹³C (75 MHz, CDCl₃) δ (75/25 mixture of rotamers): 19.6 and 21.1 (CH₃ iPr), 32.2 (CH₂ Bn), 36.9 and 38.1 (CH₂), 46.6 and 48.6 (CH), 67.4 and 68.0 (CH₂), 114.0 (C), 116.4 (CH), 127.0 (CH Bn), 128.7 (CH Bn), 128.9 (CH Bn), 132.3 (CH), 135.2 (C Bn), 156.7 and 156.9 (C), 167.6 (C), 169.4 (C), 176.1 (C). Anal. Calcd. for C₂₁H₂₂N₃O₃Br: C, 56.77; H, 4.99; N, 9.46. Found: C, 57.04; H, 5.11; N, 9.28.

Compound 31 as a colorless oil (59 mg, 61% from N-[2-(4-bromophenoxy)acetyl]-N-isopropyl glycine): ¹H (300 MHz, CDCl₃) δ (80/20 mixture of rotamers): 1.13 and 128 (d, J=6.8 Hz, 6H), 4.34 and 4.77 (hept, J=6.8 Hz, 1H), 4.56 and 4.65 (s, 2H), 4.78 (s, 2H), 6.84 (d, J=9.0 Hz, 2H), 7.40 (d, J=9.0 Hz, 2H). ¹³C (75 MHz, CDCl₃) δ: 21.2 (CH₃), 34.4 (CH₂), 37.0 (CH₂), 48.7 (CH), 67.5 (CH₂), 114.1 (C), 116.5 (CH), 132.4 (CH), 156.9 (C), 167.1 (C), 167.8 (C), 177.3 (C). HRMS (EST) calcd for C₁₅H₁₇N₃O₃ClBrNa [(M+Na)⁺] 424.0040, found 424.0040. Anal. Calcd. for C₁₅H₁₇N₃O₃ClBr: C, 48.93; H, 4.93; N, 11.41. Found: C, 48.88; H, 4.94; N, 11.13.

Compound 32 as a white solid (95 mg, 99%): Mp 114° C. ¹H (300 MHz, CDCl₃) δ (70/30 mixture of rotamers): 1.21 and 1.35 (d, J=6.8 Hz, 6H), 4.41 and 4.87 (hept, J=6.8 Hz, 1H), 4.79 and 4.88 (s, 2H), 4.83 and 4.89 (s, 2H), 6.80-6.90 (m, 2H), 7.27-7.38 (m, 2H), 7.51-7.70 (m, 3H), 7.87-8.10 (m, 2H), 8.13-8.24 (m, 1H), 8.88 (d, J=8.1 Hz, 1H). ¹³C (75 MHz, CDCl₃) δ (70/30 mixture of rotamers): 19.8 and 21.3 (CH₃), 37.0 and 38.3 (CH₂), 46.9 and 48.7 (CH), 67.7 and 68.3 (CH₂), 114.0 (C), 116.4 and 116.5 (CH), 123.6 (CH), 125.0 (CH), 126.2 (CH), 126.3 (CH), 127.6 and 127.8 (CH), 128.6 and 128.8 (CH), 129.5 and 129.7 (CH), 130.5 (C), 131.9 (C), 132.5 (CH), 133.8 (C), 157.0 (C), 167.8 (C), 168.7 (C), 175.3 (C). HRMS (ESI) calcd for C₂₄ H₂₂N₃O₃BrNa [(M+Na)⁺] 502.0742, found 502.0739. Anal. Calcd. for C₂₄H₂₂N₃O₃Br: C, 48.93; H, 4.93; N, 11.41. Found: C, 48.88; H, 4.94; N, 11.13.

Compound 25 as a white solid (74 mg, 91% from N-[2-(4-bromophenoxy)acetyl]-N-isopropyl glycine): Mp 135° C. ¹H (300 MHz, CDCl₃) δ (70/30 mixture of rotamers): 1.16 and 1.30 (d, J=6.8 Hz, 6H), 3.94 (s, 6H), 4.38 and 4.80 (hept, J=6.8 Hz, 1H), 4.69 and 4.79 (s, 2H), 4.80 and 4.85 (s, 2H), 6.80-6.89 (m, 2H), 6.94 (d, J=8.3 Hz, 1H), 7.31-7.40 (m, 2H), 7.48-7.65 (m, 2H). ¹³C (75 MHz, DMSO-d₆) δ (70/30 mixture of rotamers): 19.7 and 21.2 (CH₃ iPr), 36.9 and 38.2 (CH₂), 46.8 and 48.7 (CH), 56.0 (OCH₃), 67.7 and 68.2 (CH₂), 109.7 (CH), 111.0 (CH), 114.0 (CH), 116.5 (CH), 118.5 and 119.1 (C), 121.1 (C), 132.4 (CH), 149.1 and 149.2 (C), 151.5 and 151.9 (C), 156.9 and 157.1 (C), 167.7 (C), 168.1 and 168.4 (C), 175.9 and 176.0 (C). HRMS (ESI) calcd for C₂₂H₂₄N₃O₅BrNa [(M+Na)⁺] 512.0797, found 512.0792. Anal. Calcd. for C₂₂H₂₄N₃O₅Br: C, 48.93; H, 4.93; N, 11.41. Found: C, 48.88; H, 4.94; N, 11.13.

Compound 24 as a white solid (84 mg, 87% from N-[2-(4-bromophenoxy)acetyl]-N-isopropyl glycine): Mp 87° C. ¹H (300 MHz, CDCl₃) δ (75/25 mixture of rotamers): 1.17 and 1.31 (d, J=6.9 Hz, 6H), 4.40 and 4.81 (hept, J=6.9 Hz, 1H), 4.70 (s, 2H), 4.80 and 4.85 (s, 2H), 6.80-6.89 (m, 2H), 7.00-7.10 (m, 4H), 7.15-7.20 (m, 1H), 7.32-7.42 (m, 4H), 7.95 (d, J=9.0 Hz, 2H). ¹³C (75 MHz, CDCl₃) δ (75/25 mixture of rotamers): 19.7 and 21.2 (CH₃), 36.9 and 38.3 (CH₂), 46.9 and 48.7 (CH), 67.6 and 68.3 (CH₂), 114.0 (C), 116.5 (CH), 118.3 (CH), 119.8 (CH), 121.1 (C), 124.2 and 124.3 (CH), 129.2 (CH), 130.0 (CH), 132.4 (CH), 155.9 and 156.0 (C), 157.0 (C), 160.2 and 160.6 (C), 167.7 (C), 167.8 (C), 176.1 (C). HRMS (EST) calcd for C₂₆H₂₄N₃O₄BrNa [(M+Na)⁺] 544.0848, found 544.0849. Anal. Calcd. for C₂₆H₂₄N₃O₄Br: C, 48.93; H, 4.93; N, 11.41. Found: C, 48.88; H, 4.94; N, 11.13.

General Procedure for the Preparation of Oxadiazoles 33-35

To a solution of oxadiazole 31 (1 eq.) in dimethylformamide (0.25 M) was added the appropriate amine (3.7 eq.). After stirring for 18 h at room temperature, the resulting mixture was concentrated in vacuo, diluted by aqueous 0.1 M sodium hydroxyde and extracted by ethyl acetate. The resulting organic phase was dried over sodium sulphate and concentrated in vacuo. After addition of methanolic 2.5 M hydrochloric acid (1.1 eq) to a solution of the crude residue in methylene chloride (1.5 mL for 0.1 mmol of product) and concentration in vacuo, oxadiazoles 33-35 were afforded.

Compound 33 as a white solid (70 mg, 97%): Mp 110° C. ¹H (300 MHz, CDCl₃) δ: 1.30 (d, J=6.3 Hz, 6H), 3.10-3.50 (m, 4H), 3.90-4.15 (m, 4H), 4.30-4.34 (m, 3H), 4.63 (s, 2H), 4.77 (s, 2H), 6.81 (d, J=8.9 Hz, 2H), 7.37 (d, J=8.9 Hz, 2H). ¹³C (75 MHz, CDCl₃) δ: 21.3 (CH₃ iPr), 37.4 (CH₂), 48.8 (CH), 50.2 (CH₂), 51.3 (CH₂), 63.8 (CH₂), 67.3 (CH₂), 114.1 (C), 116.4 (CH), 132.5 (CH), 156.8 (C), 161.6 (C), 167.9 (C), 178.3 (C). HRMS (ESI) calcd for C₁₉H₂₅N₄O₄BrNa [(M+Na)⁺] 475.0957, found 475.0953 and calcd for C₂₉H₂₆N₄O₄Br [(M+H)⁺] 453.1137, found 453.1133. Anal. Calcd. for C₁₅H₁₇N₃O₃ClBr: C, 48.93; H, 4.93; N, 11.41. Found: C, 48.88; H, 4.94; N, 11.13.

Compound 34 as a slightly yellow solid (67 mg, 63%): Mp 168° C. ¹H (300 MHz, CDCl₃) δ: 1.29 (d, J=6.3 Hz, 6H), 1.48 (t J=6.7 Hz, 6H), 2.26 (s, 1H), 2.86-3.31 (m, 4H), 4.24-4.40 (m, 1H), 4.35 (s, 2H), 4.61 (s, 2H), 4.75 (s, 2H), 6.79 (d, J=8.9 Hz, 2H), 7.34 (d, J=8.9 Hz, 2H). ¹³C (75 MHz, CDCl₃) δ: 9.8 (CH₃ Et), 21.3 (CH₃ iPr), 37.4 (CH₂), 42.9 (CH₂), 48.7 (CH₂), 48.8 (CH), 67.2 (CH₂), 114.1 (C), 116.4 (CH), 132.4 (CH), 156.8 (C), 161.4 (C), 167.9 (C), 178.3 (C). HRMS (EST) calcd for C₁₉H₂₈N₄O₃BrNa [(M+Na)⁺] 461.1164, found 461.1167. Anal. Calcd. for C₁₅H₁₇N₃O₃ClBr: C, 48.93; H, 4.93; N, 11.41. Found: C, 48.88; H, 4.94; N, 11.13.

Compound 35 as a slightly yellow solid (141 mg, 99%): Mp 144° C. ¹H (300 MHz, CDCl₃) δ: 1.31 (d, J=6.3 Hz, 6H), 1.70-2.40 (m, 7H), 2.75-2.85 (m, 2H), 3.40-3.50 (m, 2H), 4.29 (s, 2H), 4.30 (m, 1H), 4.63 (s, 2H), 4.77 (s, 2H), 6.81 (d, J=8.9 Hz, 2H), 7.37 (d, J=8.9 Hz, 2H). ¹³C (75 MHz, CDCl₃) δ: 21.3 (CH₃ iPr), 21.4 (CH₂), 22.8 (CH₂), 37.4 (CH₂), 48.8 (CH), 50.2 (CH₂), 52.7 (CH₂), 67.2 (CH₂), 114.1 (C), 116.4 (CH), 132.5 (CH), 156.8 (C), 161.7 (C), 167.9 (C), 178.1 (C). HRMS (ESI) calcd for C₂₀H₂₇N₄O₃BrNa [(M+Na)⁺] 473.1164, found 473.1169 and calcd for C₂₀H₂₈N₄O₃Br [(M+H)⁺] 451.1345, found 451.1351. Anal. Calcd. for C₁₅H₁₇N₃O₃ClBr: C, 48.93; H, 4.93; N, 11.41. Found: C, 48.88; H, 4.94; N, 11.13.

What is claimed is:

1. A method for treating and/or ameliorating and/or preventing a disease or a disorder, the method comprising administering to an individual in need thereof an effective amount of a medicament comprising at least one nitrogen heterocycle derivative of formula (I): 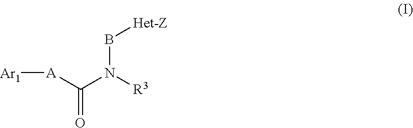
wherein Het represents a triazole or an oxadiazole radical, optionally substituted with one or more linear or branched, saturated or unsaturated, C₁-C₄ alkyl group;
Ar₁ represents a C₆-C₁₀ aryl group substituted with at least one R group selected from the group consisting of:
H,
a halogen group,
a linear, branched or cyclic, saturated or unsaturated, C₁-C₅ alkyl group,
a linear, branched or cyclic, saturated or unsaturated, C₁-C₅ alkoxy group, and
a phenoxy group;
A represents:
a covalent bond,
a linear, branched or cyclic, saturated or unsaturated, C₁-C₅ alkylene group, or
*-X—C(R⁴R⁶)-□,
with *- representing a covalent bond with Ar₁, -□ representing a covalent bond with —C(O)—, X representing a linear or branched, saturated or unsaturated, C₁-C₅ alkylene group, or a heteroatom, and R⁴ and R⁶ being, independently of each other, selected from the group consisting of H and a linear, branched or cyclic, saturated or unsaturated, C₁-C₅ alkyl group;
B represents a linear, branched or cyclic, saturated or unsaturated, C₁-C₅ alkylene group, optionally substituted with one or more C₁-C₅ hydroxyalkyl group(s), or a C₆-C₁₀ arylene group;
R³ represents H or a linear, branched or cyclic, saturated or unsaturated, C₁-C₅ alkyl group; and
Z represents —(R⁵)ₙ—(Ar₂)ₘ, with n and m representing, independently of each other, 0 or 1, provided that at least one of n or m is 1, where
R⁵ represents a linear, branched or cyclic, saturated or unsaturated C₁-C₅ alkyl or alkylamido group, optionally comprising one or more heteroatom(s) chosen among O, N or S and being optionally substituted with one or more halogen atom(s), and
Ar₂ represents a C₅-C₁₀ aryl group substituted with at least one R group.

2. The method according to claim 1, wherein the disease or disorder is a condition mediated by proteasome activity, and the medicament is a proteasome activity modulator comprising the at least one nitrogen heterocycle derivative of formula (1).

3. The method according to claim 2, wherein the substituted C₆-C₁₀ aryl group of Ar₁ in the at least one nitrogen heterocycle derivative of formula (1) is substituted with at least one R group selected from the group consisting of
H,
a halogen group,
a linear, branched or cyclic, saturated or unsaturated, C₁-C₅ alkyl group, and
a linear, branched or cyclic, saturated or unsaturated, C₁-C₅ alkoxy group.

4. The method according to claim 3, wherein the linear, branched or cyclic, saturated or unsaturated alkyl or alkylamido group of R⁵ is a C₁-C₅ alkyl or alkylamido group, optionally comprising one or more heteroatom(s) selected from the group consisting of O, N and S, and the substituted C₆-C₁₀ aryl group of Ar₂ is a C₆-C₁₀ aryl group substituted with at least one R group.

5. The method according to claim 2, wherein Het is selected from the group consisting of an 1,2,4-oxadiazole, an 1,3,4-oxadiazole, an 1,2,5-oxadiazole, an 1,2,3-oxadiazole, an 1,2,3-triazole an 1,2,4-triazole- and a 4-methyl-1,2,4-triazole radical.

6. The method according to claim 2, wherein Het is selected from the group consisting of an 1,2,4-oxadiazole, an 1,2,5-oxadiazole, an 1,2,3-oxadiazole, an 1,2,3-triazole an 1,2,4-triazole- and a 4-methyl-1,2,4-triazole radical.

7. The method according to claim 2, wherein said nitrogen heterocycle derivative is of formula (IIA) or (IIB):

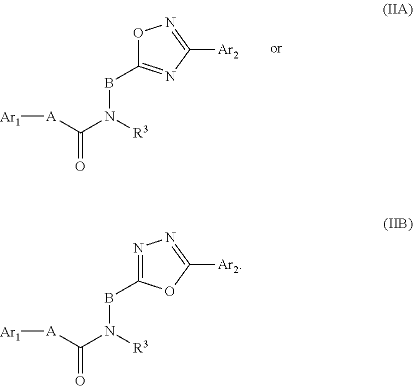

8. The method according to claim 7, wherein Ar₁ and Ar₂ represent, independently of each other, a phenyl group or a napthyl group.

9. The method according to claim 2, wherein Ar₁ is substituted with at least two R¹ groups, identical or different, said R¹ is selected from the group consisting of:
H,
a halogen group,
a linear, branched or cyclic, saturated or unsaturated, C₁-C₅ alkyl group, and
a linear, branched or cyclic, saturated or unsaturated, C₁-C₅ alkoxy group.

10. The method according to claim 9, wherein the R¹ is selected from the group consisting of:
H, Cl, Br, a methyl, an ethyl, a propyl, an iso-propyl, a n-butyl, an iso-, a sec- or a tert-butyl group, a methoxy, an ethoxy, a propoxy, or an iso-propoxy group, a n-butoxy, and an iso-, a sec- or a tert-butoxy group.

11. The method according to claim 2, wherein Ar₂ is substituted with at least two R² groups, identical or different, and said R² groups are selected from the group consisting of:
H,
a halogen group,
a linear, branched or cyclic, saturated or unsaturated, C₁-C₅ alkyl group, and
a linear, branched or cyclic, saturated or unsaturated, C₁-C₅ alkoxy group.

12. The method according to claim 11, wherein the R² groups are selected from the group consisting of:
H, a methyl, an ethyl, a propyl, an iso-propyl, a n-butyl, an iso-, a sec- or a tert-butyl group, a methoxy, an ethoxy, a propoxy or an iso-propoxy group, a n-butoxy, and an iso-, a sec- or a tert-butoxy group.

13. The method according to claim 2, wherein the R³ is selected from the group consisting of H, a methyl, an ethyl, a propyl, an iso-propyl, a n-butyl, an iso-, a sec- or a tert-butyl, a vinyl, and an allyl group.

14. The method according to claim 2, wherein A represents *-C(R⁴R⁶)—X-□, X being a methylene or O and R⁴ and R⁶, independently of each other, being H or a methyl group.

15. The method according to claim 2, wherein B is selected from the group consisting of a methylene, a hydroxymethylmethylene, an ethylene, a propylene, an iso-propylene, a phenylene, and a naphtylene group.

16. The method according to claim 2, wherein the linear, branched or cyclic, saturated or unsaturated alkyl or alkylamido group of R⁵ is a C₁-C₅ alkyl or alkylamido group, optionally comprising one or more heteroatom(s) selected from the group consisting of O, N and S and being substituted with one or more halogen atom(s), and the substituted C₆-C₁₀ aryl group of Ar₂ is a C₆-C₁₀ aryl group substituted with at least one R group selected from the group consisting of:
H,
a halogen group,
a linear, branched or cyclic, saturated or unsaturated, C₁-C₅ alkyl group, and
a linear, branched or cyclic, saturated or unsaturated, C₁-C₅ alkoxy group.

17. The method according to claim 16, wherein the C₁-C₅ alkyl or alkylamido group is substituted with one or more chlorine atoms.

18. The method according to claim 2, wherein the substituted C₆-C₁₀ aryl group of Ar₂ in the at least one nitrogen heterocycle derivative of formula (I) is substituted with at least one phenoxy group.

19. The method according to claim 18, wherein the substituted C₆-C₁₀ aryl group of Ar₁ is a C₆-C₁₀ aryl group substituted with at least one R group selected from the group consisting of:
H,
a halogen group,
a linear, branched or cyclic, saturated or unsaturated, C₁-C₅ alkyl group, and
a linear, branched or cyclic, saturated or unsaturated, C₁-C₅ alkoxy group, and
the linear, branched or cyclic, saturated or unsaturated alkyl or alkylamido group of R⁵ is a C₁-C₅ alkyl or alkylamido group optionally comprising one or more heteroatom(s) selected from the group consisting of O, N and S.

20. The method according to claim 2, wherein said nitrogen heterocycle derivative is of formula (IIIA): 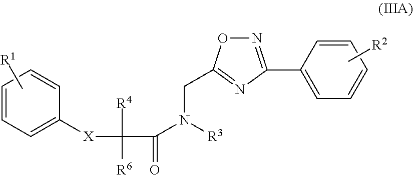
wherein
R¹, R², R³, R⁴ and R⁶ are as defined in the following table:

| R¹ | R² | R³ | R⁴, R⁶ | X |
|---|---|---|---|---|
| p-CH(CH₃)₂ | p-CH₃ | —CH(CH₃)₂ | H | O |
| p-CH(CH₃)₂ | m-CH₃ | —CH(CH₃)₂ | H | O |
| p-CH₂CH₃ | m-CH₃ | —CH(CH₃)₂ | H | O |
| p-Br | m-CH₃ | —CH(CH₃)₂ | H | O |
| p-O—CH₃ | m-CH₃ | —CH(CH₃)₂ | H | O |
| p-O—CH₃ | H | —CH(CH₃)₂ | H | O |
| p-CH₂—CH₃ | p-O—CH₃ | —CH(CH₃)₂ | H | O |
| H | p-O—CH₃ | —CH₂—CH═CH₂ | H | O |
| H | p-O—CH₃ | —CH(CH₃)₂ | H | O |
| p-CH₃ | p-O—CH₃ | —CH(CH₃)₂ | H | O |
| p-CH₃, o-Br | p-O—CH₃ | —CH(CH₃)₂ | H | O |
| p-Br | p-O—CH₃ | —CH(CH₃)₂ | H | O |
| m-CH₃ | p-O—CH₃ | —CH(CH₃)₂ | H | O |
| H | p-O—CH₃ | —CH(CH₃)₂ | H, | O |
| | | | —CH₃ | |
| m-CH₃ | m-CH₃ | —CH(CH₃)₂ | H | O |
| p-O—CH₃ | p-CH₃ | —CH(CH₃)₂ | H | O |
| H | p-O—CH₃ | —CH(CH₃)₂ | H | —CH₂ |
| p-CH₃ | H | —CH₂—CH═CH₂ | H | O |
| p-Br | p-O—Ph | —CH(CH₃)₂ | H | O |
| p-Br | p-O—CH₃ | —CH(CH₃)₂ | H | O |
| | m-O—CH₃ | | | |

or of formula (IIIB) 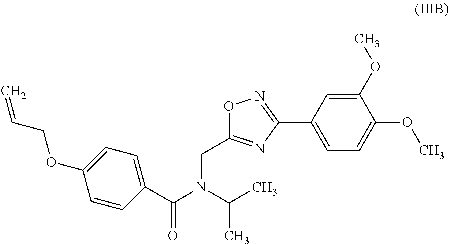
or of formula (IIIC) 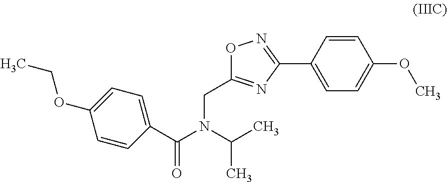
or of formula (IIID) 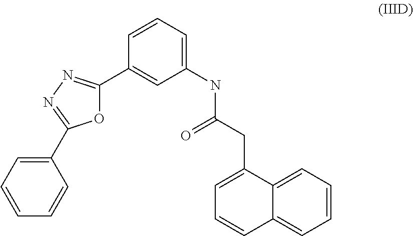
or of formula (IIIE) 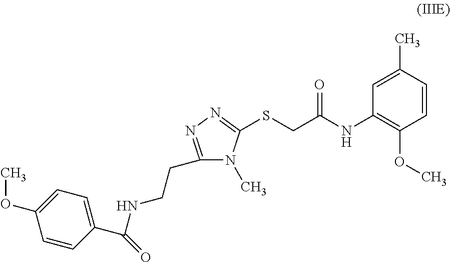
or of formula (IIIF) 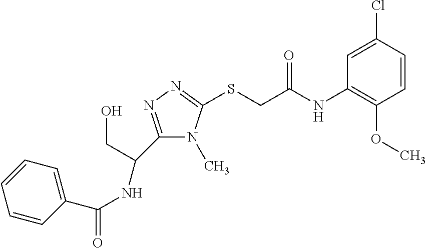
(IIIF).

21. The method according to claim 2, wherein said nitrogen heterocycle derivative is of formula (IV): 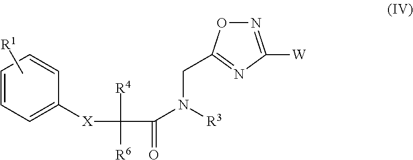
wherein:
R₁ represents a halogen group;
R₃ represents H or a linear or branched C₂-C₄ alkyl group;
R₄ and R₆ represent, independently of each other, H or a methyl group;
X represents a heteroatom selected from the group consisting of O and N; and
W is a linear, branched or cyclic, saturated or unsaturated, C₁-C₅ alkyl or alkylamido group, optionally comprising one or more heteroatom(s) selected from the group consisting of O, N and S, or a C₆-C₁₀ alkylaryl group.

22. The method according to claim 21, wherein W is a benzyl group.

23. The method according to claim 21, wherein the linear, branched or cyclic, saturated or unsaturated, C₁-C₅ alkyl or alkylamido group of W is substituted with one or more halogen atom(s).

24. The method according to claim 21, wherein said nitrogen heterocycle derivative is of formula (IV), wherein R¹, R⁴, R⁶ and W are as defined in the following table:

| R1 | R3 | R4, R6 | X | W |
|---|---|---|---|---|
| p-Br | —CH(CH₃)₂ | H | O | —CH₃ |
| p-Br | —CH(CH₃)₂ | H | O | —CH(CH₃)₂ |
| p-Br | —CH(CH₃)₂ | H | O | —C(CH₃)₃ |
| p-Br | —CH(CH₃)₂ | H | O | —CH₂-Ph |
| p-Br | —CH(CH₃)₂ | H | O | —CH₂-Cl |
| p-Br | —CH(CH₃)₂ | H | O | 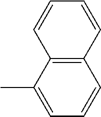 |
| p-Br | —CH(CH₃)₂ | H | O | 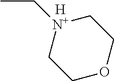 |
| p-Br | —CH(CH₃)₂ | H | O | 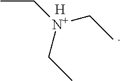 |

25. The method according to claim 2, wherein the condition mediated by proteasome activity is a disease condition selected from the group consisting of cancers, immunological diseases, auto-immune diseases, allograft rejections, viral diseases, parasitic diseases, bacterial infections, inflammatory diseases, cardiac diseases, ischemic strokes, muscular dystrophies, muscle wasting, traumatisms, burns, and disease conditions associated with aging.

26. A method for the prevention and/or treatment of skin aging, the method comprising administering to an individual in need thereof an effective amount of a cosmetic composition comprising at least one nitrogen heterocycle derivative of formula (I): 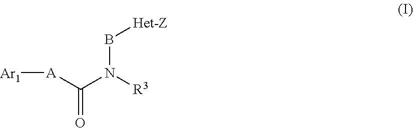
wherein Het represents a triazole or an oxadiazole radical, optionally substituted with one or more linear or branched, saturated or unsaturated, C₁-C₄ alkyl group;
Ar₁ represents a C₆-C₁₀ aryl group substituted with at least one R group selected from the group consisting of:
H,
a halogen group,
a linear, branched or cyclic, saturated or unsaturated, C₁-C₅ alkyl group,
a linear, branched or cyclic, saturated or unsaturated, C₁-C₅ alkoxy group, and
a phenoxy group;
A represents:
a covalent bond,
a linear, branched or cyclic, saturated or unsaturated, C₁-C₅ alkylene group, or
*-X—C(R⁴R⁶)-□,
with *- representing a covalent bond with Ar₁, -□ representing a covalent bond with —C(O)—, X representing a linear or branched, saturated or unsaturated, C₁-C₅ alkylene group, or a heteroatom, and R⁴ and R⁶ being, independently of each other, selected from the group consisting of H and a linear, branched or cyclic, saturated or unsaturated, C₁-C₅ alkyl group;
B represents a linear, branched or cyclic, saturated or unsaturated, C₁-C₅ alkylene group, optionally substituted with one or more C₁-C₅ hydroxyalkyl group(s), or a C₆-C₁₀ arylene group;
R³ represents H or a linear, branched or cyclic, saturated or unsaturated, C₁-C₅ alkyl group; and
Z represents —(R⁵)ₙ—(Ar₂)ₘ, with n and m representing, independently of each other, 0 or 1, provided that at least one of n or m is 1, where
R⁵ represents a linear, branched or cyclic, saturated or unsaturated C₁-C₅ alkyl or alkylamido group, optionally comprising one or more heteroatom(s) chosen among O, N or S and being optionally substituted with one or more halogen atom(s), and
Ar₂ represents a C₅-C₁₀ aryl group substituted with at least one R group.

27. A kit comprising (i) at least one nitrogen heterocycle derivative of formula (I): 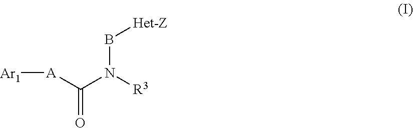
wherein Het represents a triazole or an oxadiazole radical, optionally substituted with one or more linear or branched, saturated or unsaturated, C₁-C₄ alkyl group;
Ar₁ represents a C₆-C₁₀ aryl group substituted with at least one R group selected from the group consisting of:
H,
a halogen group,
a linear, branched or cyclic, saturated or unsaturated, C₁-C₅ alkyl group,
a linear, branched or cyclic, saturated or unsaturated, C₁-C₅ alkoxy group, and
a phenoxy group;
A represents:
a covalent bond,
a linear, branched or cyclic, saturated or unsaturated, C₁-C₅ alkylene group, or
*-X—C(R⁴R⁶)-□,
with *- representing a covalent bond with Ar₁, -□ representing a covalent bond with —C(O)—, X representing a linear or branched, saturated or unsaturated, C₁-C₅ alkylene group, or a heteroatom, and R⁴ and R⁶ being, independently of each other, selected from the group consisting of H and a linear, branched or cyclic, saturated or unsaturated, C₁-C₅ alkyl group;
B represents a linear, branched or cyclic, saturated or unsaturated, C₁-C₅ alkylene group, optionally substituted with one or more C₁-C₅ hydroxyalkyl group(s), or a C₆-C₁₀ arylene group;
R³ represents H or a linear, branched or cyclic, saturated or unsaturated, C₁-C₅ alkyl group; and
Z represents —(R⁵)ₙ—(Ar₂)ₘ, with n and m representing, independently of each other, 0 or 1, provided that at least one of n or m is 1, where
R⁵ represents a linear, branched or cyclic, saturated or unsaturated C₁-C₅ alkyl or alkylamido group, optionally comprising one or more heteroatom(s) chosen among O, N or S and being optionally substituted with one or more halogen atom(s), and
Ar₂ represents a C₆-C₁₀ aryl group substituted with at least one R group;
and (ii) at least one agent useful for the prevention and/or the treatment of a cancer condition, said agent being different from said nitrogen heterocycle derivative of formula (I).
